(19) United States
(12) Patent Application Publication
(10) Pub. No.: US 2020/0105392 A1
(43) Pub. Date: Apr. 2, 2020

(54) HEALTHCARE ECOSYSTEM METHODS, SYSTEMS, AND TECHNIQUES

(71) Applicant: Symptos LLC, Chicago, IL (US)

(72) Inventors: Demetrios G. Karkazis, Park Ridge, IL (US); John Hatzigiannis, Park Ridge, IL (US)

(21) Appl. No.: 16/586,862

(22) Filed: Sep. 27, 2019

Related U.S. Application Data
(60) Provisional application No. 62/738,992, filed on Sep. 28, 2018.

(51) Int. Cl. G16H 20/10; G16H 70/40; G06Q 30/02

(52) U.S. Cl. CPC G16H 20/10; G06Q 30/0283; G16H 70/40

(57) ABSTRACT

Methods, systems, and techniques for real-time medication pricing, optimizing purchase options, analyzing medication conflicts, and optimizing medical provider options are provided. Example embodiments provide a Healthcare Advisory System (“HAS”), which enables users to select their optimal medication purchase options and enables discount drug provider (“DDPs”) to capture or retain sales that they otherwise would have lost due to price competition. In one embodiment, the HAS includes a HAS server, drug supply chain intermediary systems, pharmacy benefit manager systems, insurance provider systems, and client devices. These components cooperate to reduce computational expensive trend-based analytics that DDPs otherwise would employ to achieve worse results, thereby improving the computational efficiency of the enhanced computer- and network-based methods, techniques, and systems.

### CROSS-REFERENCE TO RELATED APPLICATION

This application claims priority from U.S. Patent Application No. 62/738,992 filed Sep. 28, 2018, the contents of which application is incorporated herein by reference in its entirety.

### TECHNICAL FIELD

The present disclosure relates to methods, techniques, and systems for real-time medication pricing and, in particular, to methods, techniques, and systems for real-time medication pricing that facilitates reducing computational expenses while providing price competition to retain a user's medication purchases.

### BACKGROUND

Typical consumers of medications have multiple purchase options when purchasing a medication. For example, a consumer can purchase a name brand or a generic version of the medication. In other examples, the consumer can purchase the medication through the consumer's insurance plan or another medication plan, e.g., a drug discount plan, or the consumer can purchase the medication out-of-pocket by selecting a purchase option that is outside of the consumer's medication plan. If the consumer selects a purchase option that is outside of the consumer's medication plan, one or more entities associated with the medication plan, e.g., a pharmaceutical benefits manager or a payor such as the consumer's employer or insurance plan, miss the opportunity to profit from the consumer's purchase of the medication.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 is an overview block diagram of an example healthcare advisory system.

FIG. 2 is an example data flow diagram using Yourdon/Coad notation, showing how data flows between components of an example healthcare advisory system.

FIG. 3 is an example block diagram of an example healthcare advisory module of an example healthcare advisory server of an example healthcare advisory system.

FIG. 4 is an example flow diagram of an overview of logic performed by an example healthcare advisory system server.

FIG. 5 is an initial (home) display screen of an example user interface of an example healthcare advisory system.

FIG. 6 is a medication overview screen of an example user interface of an example healthcare advisory system.

FIG. 7 is a warnings screen of an example user interface of an example healthcare advisory system.

FIG. 8 is an order screen of an example user interface of an example healthcare advisory system.

FIG. 9 is a medical provider results screen of an example user interface of an example healthcare advisory system.

FIG. 10 is a medical provider comparison screen of an example user interface of an example healthcare advisory system.

FIG. 11 is a medical provider results screen of an example user interface of an example healthcare advisory system.

FIG. 12 is a medical provider comparison screen of an example user interface of an example healthcare advisory system.

FIG. 13 is a prescription image upload screen of an example user interface of an example healthcare advisory system.

FIG. 14 is a symptoms input screen of an example user interface of an example healthcare advisory system.

FIG. 15 is a symptoms alert and tracker screen of an example user interface of an example healthcare advisory system.

FIG. 16 is a medical history screen of an example user interface of an example healthcare advisory system.

FIG. 17 is an account management screen of an example user interface of an example healthcare advisory system.

FIG. 18 is an emergency screen of an example user interface of an example healthcare advisory system.

FIG. 19 is an example block diagram of an example healthcare advisory system server of an example healthcare advisory system.

FIG. 20 is an example flow diagram of real-time pricing advisory logic performed by an example healthcare advisory server.

FIG. 21 is an example medication price information data model generated by an example healthcare advisory server.

FIG. 22 is an example flow diagram of price information analysis logic performed by an example healthcare advisory server.

FIG. 23 is an example flow diagram of medication analysis logic performed by an example healthcare advisory server.

FIG. 24 is an example flow diagram of symptoms analysis logic performed by an example healthcare advisory server.

FIG. 25 is an example flow diagram of medical providers analysis logic performed by an example healthcare advisory server.

FIG. 26 is an example flow diagram of generate and provide historical information logic performed by an example healthcare advisory server.

### DETAILED DESCRIPTION

Embodiments described herein provide enhanced computer- and network-based methods, techniques, and systems for real-time medication pricing, optimizing purchase options, analyzing medication conflicts, and optimizing medical provider options. Example embodiments provide a Healthcare Advisory System (“HAS”), which enables users to obtain real-time medication prices, optimize medication purchases, avoid medication conflicts, and optimize medical provider selections. For example, a user may desire to evaluate prices for a medication. In response, an example HAS can obtain both in-network and out-of-network prices for name-brand and generic versions of the medication, optionally creates opportunities for real-time price competition, and can provide the user with real-time prices of the medication from various sources. In some instances, this allows the HAS to provide “stickiness” for a medication provider because it enables the provide to compete real-time to retain a possible purchase. In some examples, the HAS evaluates historical medical information and medication plan information associated with the user to notify the user which medication purchase option is predicted to best suit the user in a single purchase instance or over a longer term (for example, a fiscal year, a remainder of a fiscal year, or other time periods). In some examples, the HAS can evaluate the medication based on medical information associated with the user to generate an alert if a medication conflict is detected and optionally suggests an alternative medication.

In addition, a user may desire to find a medical provider to treat particular symptoms or a known illness. An example HAS optionally evaluates the user's symptoms to notify the user of a candidate illness and can notify the user of one or more candidate medical providers that are predicted to be optimal for the user. In some examples, the HAS provides filtered historical information of the user (for example, medical, geographical, or other information) to one or more target entities (for example, a selected medical provider) based on an evaluation of characteristics of the target entity and optionally user preferences or other data.

Example embodiments provide a Healthcare Advisory System that provides improvements over prior techniques in that it facilitates directly notifying a system of a pharmacy benefits manager when the user actually intends to deviate from the user's medication plan, thereby reducing the computationally expensive trend-based analytics that such systems may employ to predict whether the user is expected to deviate from the medication plan. Accordingly, the HAS facilitates reducing computational expenses, facilitates increasing accuracy of deviation information obtained by the computing system and, by providing real-time pricing and optional price competition, facilitates increasing the retention of potentially deviating users.

In addition, example Healthcare Advisory Systems overcome the challenges of prior computer implementations used for real-time medication pricing, optimizing purchase options, analyzing medication conflicts, or optimizing medical provider options by incorporating artificial intelligence, e.g., machine learning, techniques where and when desired. Artificial intelligence (“AI”) can be incorporated by components of the HAS to perform one or more of the activities described in this disclosure, such as each instance of discovery, learning, or generation of information, rules, algorithms, or other activities described in this disclosure. Other uses of Al are contemplated.

For the purposes of this disclosure, the term “real time” or “real-time” refers to almost real time, near real time, or time that is perceived by a user as substantially simultaneously responsive to activity. The term “medication conflicts” includes adverse drug interactions, side effects, drug allergies, and other adverse drug effects, e.g., dystonic reactions. The term “medications” refers to prescription medications or over-the-counter medications, including supplements. In the context of medication conflicts, the term “medications” refers to prescription medications, over-the-counter medications, supplements, or recreational drugs. The term “thresholds” refers to predefined thresholds, user-selected thresholds, user-defined thresholds, or thresholds determined otherwise. The term “rules” refers to predefined rules, determined rules, or learned rules. The term “predefined rules” refers to rules defined by one or more predefined scripts or configuration files. The term “learned rules” refers to rules defined by one or more artificial intelligence algorithms, e.g., machine learning algorithms, that have undergone a learning or calibration process, e.g., recursive learning based on one or more training data sets. The term “medical information” refers to medication information or non-medication medical information, including, for example, medical provider notes, medical events (e.g., injuries, illnesses, allergic reactions, or other events), illness information, etc. Unless context clearly indicates otherwise, the term “medical” as a descriptor, e.g., medical history, refers to the term being described in association with medication and non-medication. The term “Discount Drug Provider” or “DDP” refers to an example drug supply chain intermediary. Other examples of drug supply chain intermediaries include pharmacies, pharmacy discount programs, pharmaceutical manufacturers, and pharmaceutical wholesalers. This disclosure should be interpreted to disclose each embodiment involving drug discount providers, once with the term “Discount Drug Provider” as expressly written, once with the term “drug supply chain intermediary” in its place, and again for each of the above-listed examples of drug supply chain intermediaries. The term “pharmacy” refers to a mail order pharmacy, a specialty pharmacy, or a community pharmacy. The term “designated” refers to predefined, user-selected, or user-defined.

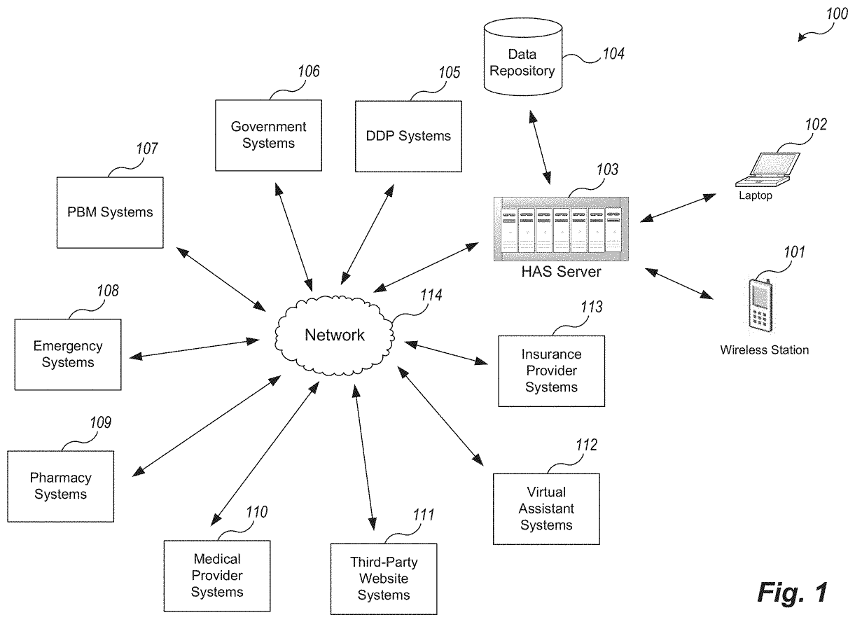

FIG. 1 is an overview block diagram of an example Healthcare Advisory System (“HAS”). In one example embodiment, the HAS installation (environment or topology) comprises one or more functional components/modules that work together to provide real-time medication pricing, purchase option optimization, medication conflicts analysis, and medical provider option optimization. Healthcare Advisory System 100 comprises a platform having one or more client devices, e.g., wireless station 101, laptop 102, or other devices, that are communicably coupled to a HAS server 103. HAS server 103 also communicably couples to one or more data repositories, e.g., a data repository 104. In addition, one or more systems are accessible to the client devices 101 and 102 or the HAS server 103 via one or more application programming interfaces (APIs). The one or more accessible systems may include one or more Discount Drug Provider (“DDP”) systems 105, government systems 106 (e.g., United States Food and Drug Administration (“FDA”), National Institute of Health (“NIH”), Medicare, or Veterans Administration (“VA”) systems), Pharmacy Benefits Manager (“PBM”) systems 107, emergency systems 108 (e.g., emergency response systems or first responder systems), pharmacy systems 109, medical provider systems 110, third-party website systems 111, virtual assistant systems 112, and/or insurance provider systems 113. Each of the components of the HAS 100 may communicably couple directly or indirectly, e.g., via a network 114 (for example, the Internet), to one or more other components.

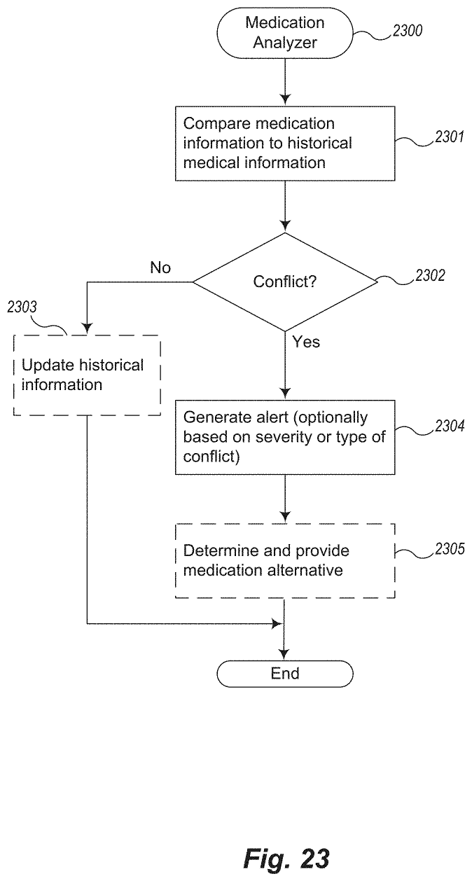

A client device, e.g., wireless station 101 or laptop 102, can initiate a healthcare advisory process involving the HAS server 103. Some healthcare advisory processes begin with the HAS server 103 obtaining medication information. The client device or the HAS server 103 may provide the medication information. The HAS server 103 analyzes the medication information to detect medication conflicts associated with the medication identified by the medication information. For example, the HAS server 103 may obtain historical information, e.g., historical medical information, associated with the user from data repository 104 and may evaluate the medication information to look for medication conflicts that the HAS server 103 determines the user is susceptible to experiencing based on the historical information. The HAS server 103 then may recommend one or more alternatives to the medication that are less likely to contribute to the medication conflict. The HAS server 103 then may revise the medication information based on the one or more alternative medications and proceed to execute the healthcare advisory process. The HAS server 103 medication analyzer functions are described further with respect to FIG. 23.

The HAS server 103 also obtains price information associated with the medication information from one or more discount drug provider (“DDP”) systems 105. The price information may include in-network and out-of-network prices of name-brand and generic versions of the medication at one or more pharmacies. The HAS server 103 facilitates price competition to revise the price information, for example, by notifying one or more of discount drug provider (“DDP”) systems 105 (e.g., one or more pharmacy benefits manager (“PBM”) systems 107) that the HAS server 103 has determined that this user is likely to purchase the medication outside of the user's medication plan, e.g., the user's pharmacy insurance plan. For example, the HAS server 103 may provide one or more DDP systems 105 one or more notifications that indicate a user is searching to fill a prescription, a current price quote from each DDP system 105 (typically with DDP names masked), and a request to match or beat the lowest price in the current price quotes. A DDP system 107, such as PBM system 107, then has the opportunity to determine and render a new price. The HAS server 103 revises the price information based on the new price. In some examples, the HAS server 103 provides the one or more of DDP systems 105, such as PBM systems 107, with the lowest price out-of-network purchase option or the lowest price out-of-network purchase option that the user is likely going to consider, e.g., the lowest price out-of-network purchase option that is closest to or within a designated distance of the user's location or a location selected by the user.

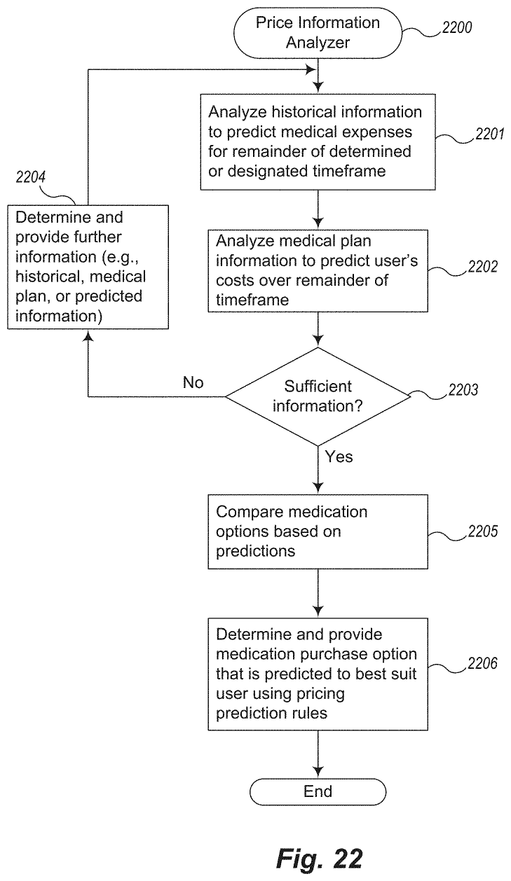

In some scenarios, the HAS server 103 analyzes the price information to determine whether a particular purchase option is predicted to be cheaper for the user over a designated timeframe, e.g., a fiscal year, a remainder of a fiscal year, or other times. For example, one purchase option may be cheaper for the user if the user has pharmaceutical expenses that are less than a threshold amount during the timeframe, and a different purchase option may be cheaper for the user if the user has pharmaceutical expenses that are greater than the threshold amount during the designated time period based on one or more characteristics of the user's insurance plan or DDP (e.g., copay amounts, coinsurance amounts, deductible amounts, or out-of-pocket maximum amounts). The HAS server 103 price information analyzer functions are described further with respect to FIG. 22.

The HAS server 103 also may analyze the status of the medication order and provides the user a notification indicating the status at one or more designated stages of the order fulfillment process. For example, the HAS server 103 may obtain an indication of the status of the prescription order in the order fulfillment process (e.g., order entered, claim adjudicated, claim rejected, order filled, pharmacist clinical check completed, pharmacist product check completed, ready for pickup or delivery, or order completed), and may notify the user when the HAS server 103 obtains one or more of the indications (e.g., a ready-for-pickup notification).

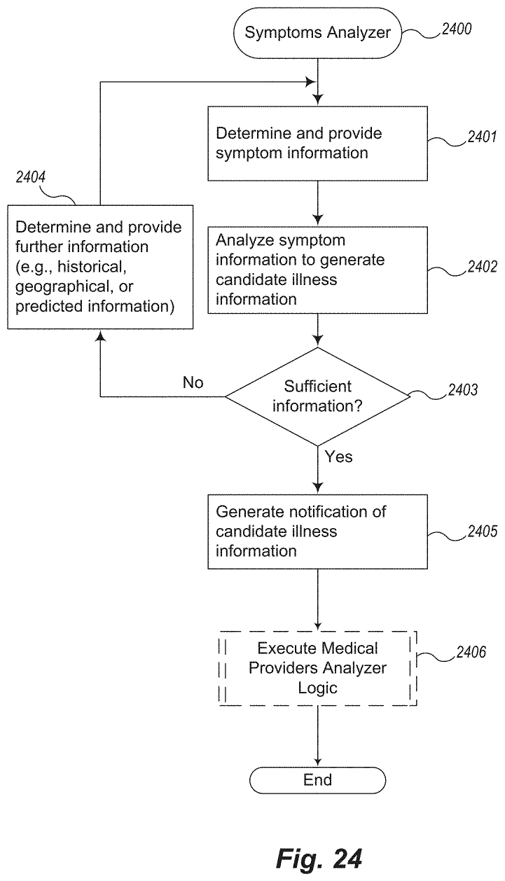

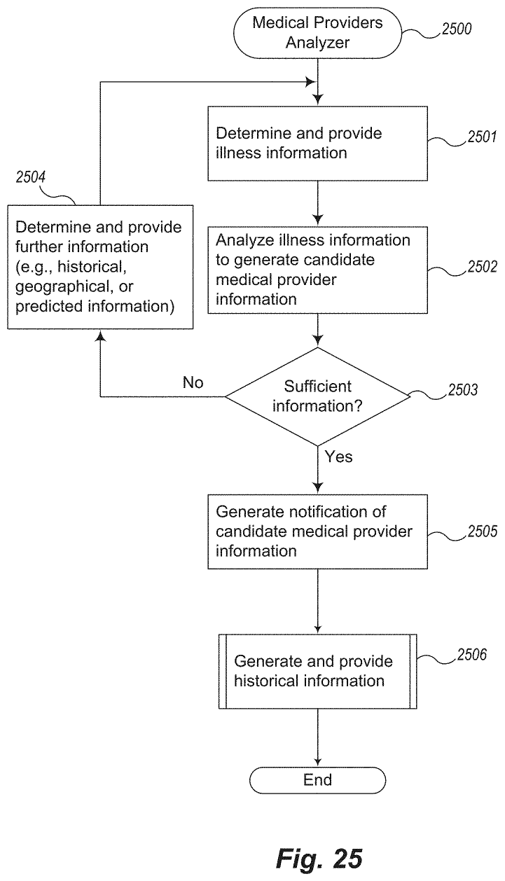

In some scenarios, the HAS server 103 facilitates generating candidate illness information based on symptom information obtained from the user and notifies the user of a candidate illness associated with the candidate illness information. The HAS server 103 symptoms analyzer functions are described further with respect to FIG. 24. Also, the HAS server 103 may generate one or more recommendations of medical providers (e.g., physicians, dentists, hospitals, or other providers) based on illness information (e.g., candidate illness information, illness information provided by the user, or others) and provide historical information to a selected medical provider. For example, the HAS server 103 may obtain medical provider information from data repository 104 based on the illness information. The HAS server 103 medical providers analyzer functions are described further with respect to FIG. 25.

Although the examples described herein often refer to the user as being a patient, e.g., the one to consume the medication or to have the symptoms or illness, the user may be a medical provider, a caretaker for a patient, a relative of a patient, or other associated user.

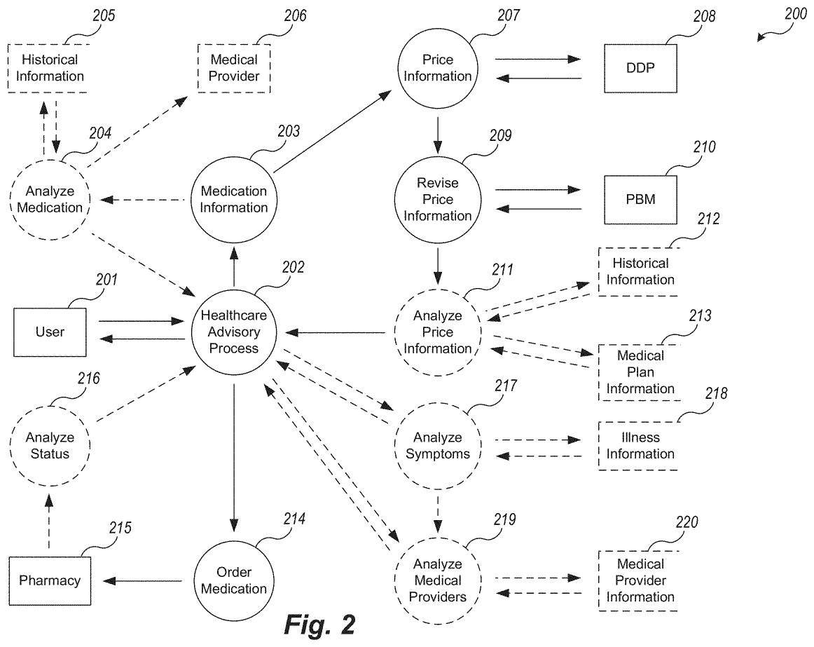

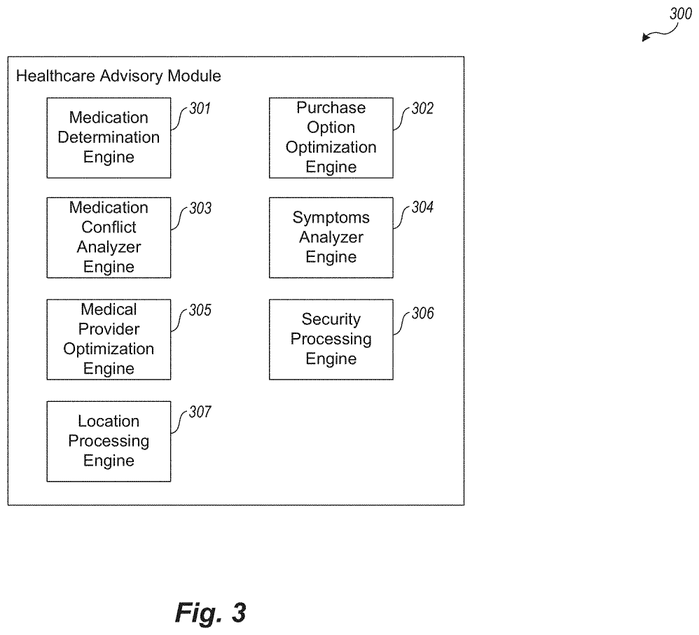

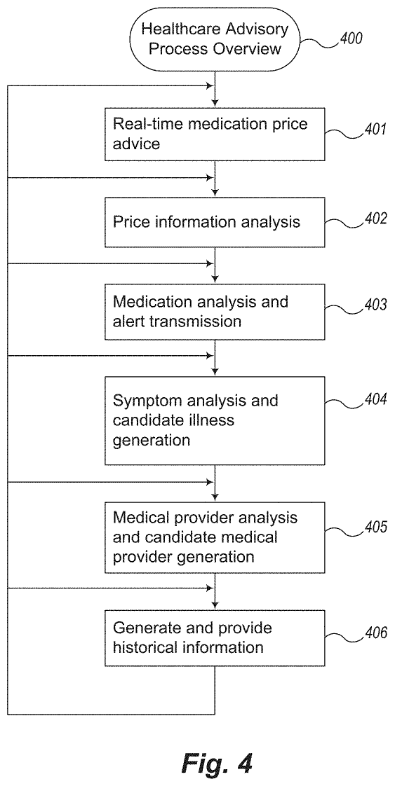

FIG. 2 is an example data flow diagram using Yourdon/Coad notation, showing how data flows between components of an example healthcare advisory system, such as the HAS 100. In FIG. 2, the enclosed rectangles represent entities outside of a HAS server, such as the HAS server 103. For example, the external entities may send or receive data to or from the HAS server and may be the sources or destinations of information entering or leaving the HAS server. The external entities may also be referred to as terminators, sources, sinks, or actors. The circles represent processes. For example, each process may change data and may produce an output. In some examples, each process performs computations, sorts data based on logic, or directs the data flow based on rules. The rectangles having an open right side represent data stores. For example, the data stores may include files or repositories that hold information for later use, such as a database table or a membership form. The arrows represent data flow. For example, each arrow may represent the route that data takes between entities, processes, or data stores. Each arrow portrays the interface between components. Optional elements of FIG. 2 are shown with dashed lines. In some examples, the HAS server performs one or more functions of one or more portions of one or more processes of FIG. 2 by executing one or more rules or portions of logic that are represented by one or more engines shown in FIG. 3 or blocks shown in FIGS. 4, 20, and 22-26.

FIG. 3 is an example block diagram of an example Healthcare Advisory Module (“HAM”) of an example healthcare advisory server of an example healthcare advisory system. For example, the HAS server 103 may incorporate the HAM 300 to perform one or more of the functions described herein. The HAM 300 comprises one or more functional components or engines that work together and with the HAS components (e.g., the components of the HAS 100 shown in FIG. 1) to execute one or more healthcare advisory processes, such as real-time medication pricing, purchase option optimization, medication conflicts analysis, and medical provider option optimization. One or more portions of one or more healthcare advisory processes may include the acts and logic described with regard to FIG. 4 and other figures. For example, the HAM 300 may comprise one or more medication determination engines 301, purchase option optimization engines 302, medication conflict analyzer engines 303, symptoms analyzer engines 304, medical provider optimization engines 305, security processing engines 306, and/or location process engines 307. One or more of these components/engines may or may not be present in any particular example embodiment. In some example embodiments, one or more portions of one or more components/engines may be components/engines of a client device, e.g., the wireless station 101 or the laptop 102.

The determination engine 301 is used to facilitate determining medication information associated with a particular medication, such as the medication name, brand name, manufacturer, class, formulary tier, dosage, active substances in the medication, excipients (i.e., one or more inactive substances that serve as a vehicle or medium for a drug or other active substance), general indications, particular indications, general regimens, particular regimens, efficacies, sight-of-service restrictions, etc.. For example, the medication determination engine 301 may represent one or more rules or portions of logic that, when executed by the HAS server 103, cause the HAS server 103 to perform one or more portions of the healthcare advisory process 202, the medication information process 203, the analyze medication process 204, or other logic. The portions of the rules or portions of logic associated with the medication determination engine 301 are described further with respect to FIGS. 4, 20, and 23-26.

In some examples, the medication determination engine 301 provides a user interface, e.g., a graphical user interface (“GUI”) having one or more input fields or other user interface (“UI”) controls, that facilitates obtaining medication information from the user. In some examples, the medication determination engine 301 generates one or more portions of the medication information based on one or more photographs of one or more prescriptions, prescription labels, or other things. The medication determination engine 301 may obtain the medication information directly from one or more of the medical provider systems 110, such as systems 110, or from a data repository, such as the repository 104, based on one or more generated or obtained portions of the medication information. The medication determination engine 301 may also generate one or more recommendations to employ as alternatives to one or more analyzed medications. The medication information may include the name of the prescribed medication, dosage, dosage interval, start date associated with the medication, date that the medication is expected to run out or expire, whether the medication can be refilled without another prescription, and the prescribed quantity or amount of the medication.

The option optimization engine 302 is used to facilitate providing real-time medication price advice or price information analysis. For example, the purchase option optimization engine 302 may represent one or more rules or portions of logic that, when executed by the HAS server 103, cause the HAS server 103 to perform one or more portions of the healthcare advisory process 202, the price information process 207, the revise price information process 209, the analyze price information process 211, or other processes. In some examples, logic may determine if a lower price becomes available between the start date and the expected date that the medication will expire or run out and may notify the user of the lower price. If the medication has a refill option without another prescription, the user may be notified of the lower price and provided a refill option based on the lower price. In other examples, logic may propose a reminder schedule to provide reminders to the user when it is time to take the medication. The user's medical provider that prescribed the medication may be notified when the user indicates that the user consumed the medication according to the alert or prescribed dosage interval.

In some examples, the option optimization engine 302 obtains medical plan information 213 (e.g., insurance provider information) from a data repository, such as the data repository 104, based on medication information to generate optimized price information for the user and notify the user accordingly. A rules based analysis may be applied to historical medication purchase information (e.g., price information or medication information associated with historical purchases) obtained from the user or other sources (e.g., medication plan providers or pharmacies) and to current price and medication information to determine price trends associated with the medication. If prices are determined to be increasing or decreasing, the user may be notified accordingly with an alert that suggests ordering the medication earlier than the expected date that the medication will expire or run out. The rules or logic associated with the option optimization engine 302 are described further with respect to FIGS. 4 and 22.

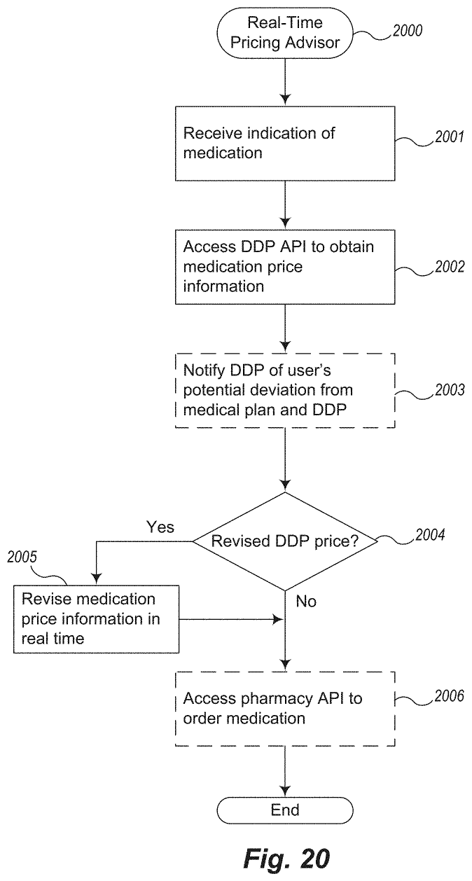

In some examples, the purchase optimization engine 302 obtains an indication of a medication, accesses an application programming interface (“API”) of one or more DDP systems 105 to obtain price information associated with the indicated medication, and notifies the user in real-time of the price information. The purchase optimization engine 302 may also facilitate price competition to revise the price information. For example, the purchase option optimization engine 302 may notify one or more of the DDP systems 105 or PBM systems 107 that the purchase option optimization engine 302 has determined that the user is likely to purchase the medication outside of the user's medication plan as discussed further with respect to FIG. 20. The purchase option optimization engine 302 may additionally or alternatively check with one or more insurance provider systems 113 associated with one or more insurance plans associated with the user to determine if the user's medication is covered by a prescription benefit plan and price information associated with the medication and the prescription benefit plan.

The purchase option optimization engine 302 may also analyze the price information to determine whether a particular purchase option is predicted to be cheaper for the user over a designated or determined timeframe. The purchase option optimization engine 302 notifies the user of the purchase option that is predicted to be cheaper over the timeframe, such as by notifying the user of one or more predicted or estimated values that led to the pricing determination. The purchase option optimization engine 302 may also facilitate the user selecting or modifying one or more portions of the historical information employed in making the determination.

In some scenarios, the purchase option optimization engine 302 provides revised price information for multiple purchase options. The purchase option optimization engine 302 optionally also notifies the user of the particular purchase option that is predicted to be cheaper for the user over the timeframe. If the purchase option optimization engine 302 obtains a user selection of a purchase option for the medication, the engine 302 orders the medication from the pharmacy associated with the selected purchase option via one or more of pharmacy systems 109. Further, the purchase option optimization engine 302 may analyze the status of the medication order and provide a notification indicating status of the order at one or more designated stages of the order fulfillment process. For example, the engine 302 may obtain from a pharmacy system 109 an indication of the status of the prescription order in the order fulfillment process such as order entered, claim adjudicated, claim rejected, order filled, pharmacist clinical check completed, pharmacist product check completed, ready for pickup or delivery, or order completed and may notify the user accordingly.

Price information may be cached, and, if a timestamp associated with a cached price indicates that the cached price information is stale, the cached price information may be replaced with updated price information, thereby reducing computational expenses required to update the price information for each user query. A learning algorithm may monitor how frequently medication prices change for each source of the price information and, taking into account computer resource expense and traffic patterns, selects times to request updated price information to reduce or minimize computational expenses during high traffic or computationally expensive times. This feature may be overridden if the price information should be updated before the next optimal time arrives.

The medication conflict analyzer engine 303 is used to facilitate detecting medication conflicts or to facilitate determining if there is an unacceptably high likelihood of the user experiencing one or more medication conflicts associated with the medication indicated by the medication information. For example, the medication conflict analyzer engine 303 may represent one or more rules or portions of logic that, when executed by the HAS server 103, cause the HAS server 103 to perform one or more portions of the healthcare advisory process 202, the analyze medication process 204, or other processes. The rules or logic associated with medication conflict analyzer engine 303 are described further with respect to FIGS. 4 and 23.

In some examples, the medication conflict analyzer engine 303 obtains historical information, e.g., historical medical information, associated with the user from data repository 104 and evaluates the historical information to search (e.g., look, examine, etc.) for one or more medication conflicts and alerts the user or one or more of medical provider systems 110 accordingly if it is determined that the user is particularly susceptible to experiencing an associated medication conflict. The medication conflict analyzer engine 303 may also recommend one or more alternatives to the medication that are less likely to contribute to a medication conflict. If an alternative medication is indicated, the medication conflict analyzer engine 303 revises the medication information (e.g., determined by the medication determination engine 301) based on the one or more alternative medications. In some examples, a concise description of the conflict may be presented to the user, and the user may be provided an option to provide the information to a medical provider (for example, the medication conflict analyzer engine 303 may initiate providing the information to the medical provider through short message service (“SMS”) or email). In some examples, the alternative medications may be presented to the user with their respective costs.

The symptoms analyzer engine 304 is used to facilitate generating candidate illness information based on symptom information obtained from the user (e.g., through a user interface) and to facilitate notifying the user of an associated candidate illness. For example, the symptoms analyzer engine 304 may represent one or more rules or portions of logic that, when executed by the HAS server 103, cause the HAS server 103 to perform one or more portions of the healthcare advisory process 202, the analyze symptoms process 217, or other actions. The rules or logic associated with symptoms analyzer engine 304 are described further with respect to FIGS. 4, 24, and 25. In some examples, the symptoms analyzer engine 304 obtains candidate illness information, e.g., symptom information, demographic information, or other information, from a data repository, such as the data repository 104, based on the symptom information obtained from the user to generate one or more candidate illnesses.

The medical provider optimization engine 305 is used to facilitate generating one or more recommendations of medical providers (e.g., physicians, dentists, hospitals, or others) based on illness information (e.g., candidate illness information, illness information provided by the user, or others). For example, the medical provider optimization engine 205 may represent one or more rules or portions of logic that, when executed by the HAS server 103, cause the HAS server 103 to perform one or more portions of the healthcare advisory process 202, the analyze price information process 211, the analyze medical providers process 219, or other actions. In some examples, the option optimization engine 302 obtains medical plan information 213 (e.g., insurance provider information or other information) from a data repository, such as the data repository 104, based on illness information 218 to generate one or more portions of medical provider information 220 or price information for the user and notify the user accordingly. The rules or logic associated with the medication determination engine 301 are described further with respect to FIGS. 4, 24, and 25. In some examples, the medical provider optimization engine 305 obtains medical provider information (e.g., physician information, dentist information, hospital information, etc.) from a data repository, such as the data repository 104, based on the illness information to generate medical provider recommendations for the user and notify the user accordingly.

The security processing engine 306 is used to facilitate providing historical information to a medical provider based on user selection of one or more recommended medical providers. For example, the security processing engine 306 may represent one or more rules or portions of logic that, when executed by the HAS server 103, cause the HAS server 103 to perform one or more portions of the healthcare advisory process 202, the analyze medication process 204, the price information process 207, the revise price information process 209, the order medication process 214, the analyze symptoms process 217, or other actions. The rules or logic associated with the security processing engine 306 are described further with respect to FIGS. 4, 23, 25, and 26. In some examples, the security processing engine 306 obtains the user's medical history information from a data repository (e.g., the data repository 104), determines that one or more portions of the medical history information are related to symptoms information, an illness, or a candidate illness, and provides the medical history information to the medical provider. The security processing engine 306 may also filter or anonymize the medical history information, for example, based on user preferences.

In some examples, information that directly or indirectly identifies the user (e.g., name, address, or other information) is stored in a data object that is separate and distinct from a data object that stores analytically interesting information, such as illness or symptom information, medication information, price information, or other information that does not identify the user. Accordingly, the analytically interesting information may be searched, analyzed, or shared without compromising anonymity of the user. One or more identifiers may indicate a relationship between the identifying information and the analytically interesting information.

The location processing engine 307 is used to facilitate generating or analyzing location information associated with the user, a patient, a medical provider, or others. For example, the location processing engine 307 may represent one or more rules or portions of logic that, when executed by the HAS server 103, cause the HAS server 103 to perform one or more portions of the healthcare advisory process 202, the price information process 207, the revise price information process 209, the analyze symptoms process 217, the analyze medical providers process 219, or other actions. The rules or logic associated with the medication determination engine 301 are described further with respect to FIGS. 4, 20, and 24-26.

In some examples, the location processing engine 307 generates location information (e.g., postal code information, city information, county information, or state information) based on geolocation information obtained from one or more positioning systems associated with a client device, for example, a global positioning system (“GPS”) receiver. The location processing engine 307 also analyzes location information associated with a client device and with illness or symptom information obtained from a data repository, such as the data repository 104, and notifies another engine, for example, the symptoms analyzer engine 304, when the location processing engine 307 determines that there is a correlation between the location information associated with the client device and the location information associated with the illness/symptom information to facilitate associating weights with one or more factors for other determinations, such as determining the likelihood that the user will deviate from the user's medical plan (e.g., a price may be lower but farther from the user which may result in a lower likelihood that the user will pursue that lower price option).

FIG. 4 is an example flow diagram of an example overview of logic performed by an example healthcare advisory system server. For example, the logic of FIG. 4 may be implemented by the HAS server 103 of FIG. 1 using a healthcare advisory module, such as the HAM 300. This logic also may be performed by different components and distributed depending on the particular configuration of the HAS.

For example, in block 401, the HAS may generate and provide real-time medication price advice. The price advice may be indicative of multiple purchase options for a medication, for example purchase options of generic and name brand versions of the medication from multiple pharmacies. Each purchase option may include a price of each version of the medication at each pharmacy indicated in the purchase options. In some cases, the HAS obtains an indication of medication that the user intends to purchase, obtains price information from one or more DDPs, and provides the price information to the user in real-time or near real-time. Optionally, the HAS facilitates price competition by notifying the one or more DDPs, such as a PBM associated with the user, that the user is likely to deviate from the user's medication plan to provide the one or more DDPs an opportunity to communicate a lower price for a version of the indicated medication. In some examples, if one or more DDPs communicate a more competitive price for one or more versions of the medication, the HAS revises the price information to include the revised price information obtained from the one or more DDPs. The user may select one of the provided purchase options, and the HAS may order or facilitate ordering the selected version of the medication from a pharmacy associated with the selected purchase option. The logic associated with block 401 is discussed further with respect to FIG. 20.

In block 402, the HAS causes analysis of price information, such as the price information generated in block 401. For example, the HAS may analyze historical medical expenses of the user to predict medical expenses of the user for the remainder of a determined timeframe, analyze the user's medication plan information to predict the user's medical costs over the remainder of the timeframe, and compare the purchase options to predict which of the purchase options will provide the lowest predicted costs for the user over the remainder of the timeframe. This analysis may take into account weightings based upon location information to determine probabilities of the various options available. The logic associated with block 402 is discussed further with respect to FIG. 22.

In block 403, the HAS analyzes the medication and, if warranted, transmits an alert. For example, the HAS may compare the obtained medication information to historical medical information associated with the user to determine one or more likelihoods that the user will experience one or more adverse reactions to the medication and, if the HAS determines that the likelihoods exceed one or more thresholds, the HAS may generate and transmit one or more alerts. An alert may be transmitted to the user or a medical provider that prescribed the indicated medication for the user. The historical medical information may include information regarding medications that the user previously used and indicates whether the user did or did not experience one or more adverse reactions, whether each adverse reaction is at a level of severity that exceeds a designated level, or one or more types of adverse reactions. The historical medical information may also include information regarding the one or more adverse reactions, the severity levels of the adverse reactions, or the types of adverse reactions. The historical medication information may also include information regarding medications or other substances (e.g., recreational drugs, foods, or other substances) that the user currently uses or is expected to use concurrently with the evaluated medication. Based on the historical medical information, the HAS generates one or more probabilities that the user will experience adverse effects or the types of adverse effects.

In some examples, the HAS compares the generated probabilities to one or more thresholds, and, if the HAS determines that they exceed the thresholds by an amount, the HAS generates an alert. In other examples, the HAS generates an alert if the user is expected to experience one or more selected types of adverse effects. If an alert is generated, the HAS server optionally determines and recommends one or more alternative medications. The logic associated with block 403 is discussed further with respect to FIG. 23.

In block 404, the HAS analyzes one or more symptoms that the user is experiencing and generates one or more candidate illnesses based on the symptoms. In some examples, the HAS provides a user interface (e.g., a GUI) that permits the user to input or select one or more symptoms that the user is experiencing, and, based on the one or more obtained inputs or selections, generates symptom information. In other examples, the HAS obtains symptom information from one or more sensors, such as from wearable devices, implanted devices, or other devices. Based on the received symptom information, the HAS generates one or more probabilities of associated one or more illnesses.

In some examples, the HAS determines the illnesses to associate with the symptom information based upon a determined number of illnesses with the highest probability as associated with the symptom information (for example, the top five illnesses that correlate to the symptoms). In other examples, the HAS dynamically determines how many of the illnesses to select as the one or more candidate illnesses based on the distribution of probabilities of their correlations to the symptoms. Accordingly, the HAS may select all or less than all of the candidate illnesses. For example, if the HAS determines that there is a large difference in probabilities between one or more of the illnesses associated with the one or more highest probabilities and the next highest-ranking illness, the HAS may select the one or more illnesses as the candidate illnesses. The HAS generates candidate illness information and provides the candidate illness information to the user, which may include, for each candidate illness, one or more of an identifier for the candidate illness, a description of the illness, a description of one or more treatments, or other factors. The logic associated with block 404 is discussed further with respect to FIG. 24.

In block 405, the HAS analyzes one or more medical providers, generates candidate medical provider information, and provides the generated candidate medical provider information to the user. For example, the HAS may determine or generate one or more portions of illness information and analyze the illness information to generate the candidate medical provider information. The illness information may be obtained from the user or may include determined or generated illness information, such as the candidate illness information. In a typical HAS, the HAS generates the candidate medical provider information for medical providers that have practice areas that cover the associated illness. The HAS may evaluate location information associated with the user or the medical providers in determining which one or more medical providers to recommend to the user. The logic associated with block 405 is discussed further with respect to FIG. 25.

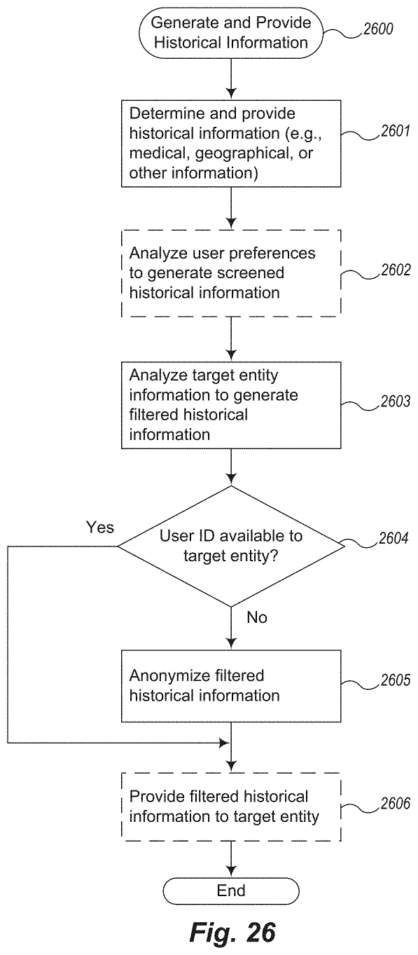

In block 406, the HAS generates and provides historical information, for example, based on a determined purpose or entity to which the historical information is intended to be provided. For example, the HAS may identify or select one or more portions of the user's medical history that the HAS determines are relevant to an impending or ongoing medical treatment and may generate the historical information based on the one or more identified or selected portions of the user's medical history. The HAS may filter and/or anonymize the generated historical information based on user or target entity preferences. The HAS may also provide the generated historical information to one or more identified target entities. In some examples, the target entity is selected by the user. The logic associated with block 406 is discussed further with respect to FIG. 26.

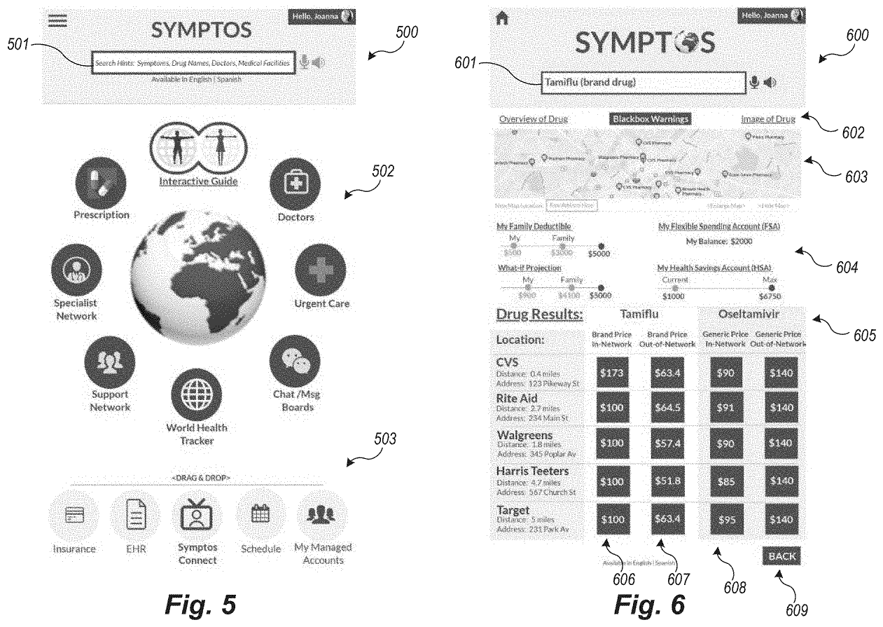

FIGS. 5-18 are example display screens from an example HAS client system. Other HAS examples may have other display screens, presented in other orders, and with other content. In some examples, one or more of the screens may be displayed on a client device, such as the wireless station 101 or the laptop 102 of FIG. 1. Many of the display screens shown in FIGS. 5-18 include user interface (“UI”) controls, which may be selectable icons or other graphical interface controls. The UI controls also may be implemented as voice commands, audibly provided options or prompts, or by haptic methods.

FIG. 5 is an initial (home) display screen of an example user interface of an example healthcare advisory system. The client device may provide an example home screen 500 by default upon executing a healthcare advisory application that runs on the client device. The displayed home screen 500 includes a search box 501, HAS process UI controls 502, and “favorite-feature” UI controls 503. The user can initiate one or more HAS processes by entering one or more terms in the search box 501 to perform a search based on the one or more entered terms. For example, using the search box 501, the user may research medications, symptoms, medical providers, illnesses, or other information. For example, searching medication information may cause a HAS server, e.g., the HAS server 103, to execute one or more portions of the logic associated with FIG. 20, 22, or 23. Searching symptoms information may cause the HAS server to execute one or more portions of logic associated with FIG. 24. Searching medical provider or illness information may cause the HAS server to execute one or more portions of the logic associated with FIG. 25.

The user can also initiate one or more portions of the HAS processes by selecting one or more process UI controls 502. For example, the HAS process UI controls 502 may include a doctors UI control, an urgent care UI control, a chat/message boards UI control, a world health tracker UI control, a support network UI control, a specialist network UI control, a prescription UI control, an interactive guide UI control, or other UI controls. In an example HAS, selecting the doctors UI control, the urgent care UI control, or the specialist network UI control may initiate one or more portions of the logic associated with FIG. 25. Selecting the world health tracker UI control may cause the HAS server to provide information that causes the client device to display a heat map that indicates the density of reported or projected cases of one or more selected illnesses or symptoms (such information may be obtained from public sources, such as Centers for Disease Control and Prevention (“CDC”), World Health Organization (“WHO”), or local health monitoring agencies). Selecting the support network UI control may cause the HAS server to provide information associated with a support network of other participating users of the HAS who also suffer from (or who are connected to others who suffer from) the same or similar symptoms or illnesses to facilitate positive collaboration. Selecting the prescription UI control may initiate one or more portions of logic associated with FIG. 20, 22, or 23. Selecting the interactive guide UI control may initiate one or more portions of logic associated with FIG. 14 with a rules based series of interactive questions to narrow and identify candidate symptoms or conditions and with rules based analytics to identify in-network and out-of-network medical providers that are available to treat the candidate symptoms or conditions based on the user's geographical location or geolocation preferences.

In an example HAS, the “favorite-feature” UI controls 503 are rearrangeable or interchangeable by the user to facilitate the user choosing which UI controls to include in “favorite-feature” UI controls 503. As shown in FIG. 5, the “favorite-feature” UI controls 503 include an insurance UI control, an electronic healthcare records (“EHR”) UI control, a connect UI control, a schedule UI control, and a “my managed accounts” UI control. Selecting one or more of the “favorite-feature” UI controls 503 causes the client device to execute one or more portions of logic, which may include obtaining or providing information or instructions from the HAS server. For example, selecting the insurance UI control may cause the client device to display one or more portions of insurance information that is associated with the user's insurance plans. The insurance information may include associate information such as one or more deductibles and progress toward each deductible, one or more out-of-pocket maximums and progress toward each out-of-pocket maximum, total expenses, itemized expenses, monthly average expenses, maximum quantities per prescription, maximum quantities per timeframe (e.g., one month), maintenance supply quantity requirements, or other values. The insurance information also may include the same or similar information for one or more of the user's DDPs. Selecting the EHR UI control may cause the client device to display one or more portions of EHR information associated with the user or an option to have the HAS share the EHR information with one or more other components in HAS, such as with other users, medical providers, or other entities. For example, the EHR information may include portions of the user's health history (including records associated with presently ongoing or impending treatments), such as medical provider visits or notes, medication prescriptions or recommendations, or other information.

Also, as another example, selecting the connect UI control may cause the client device to engage in a live communication session, e.g., live telephone, video, or text chat, with a medical provider or staff of a medical provider. Selecting the schedule UI control also may cause the client device to display one or more scheduled events associated with the user or a calendar having the user's scheduled events. For example, the scheduled events may include medical appointments, scheduled refill dates for one or more medications, scheduled times to consume medication, insurance renewal dates, or other dates. Selecting the “my managed accounts” UI control may cause the client device to display account information associated with one or more accounts that the user manages. The account information may include, for each managed account, the name of an individual associated with the managed account, insurance information associated with the individual, EHR information associated with the individual, schedule information associated with the individual, contact information associated with the individual, or others.

FIG. 6 is a medication overview screen of an example user interface of an example healthcare advisory system. An example medication purchase option screen 600 includes a search box 601, medication information UI controls 602, a purchase option location map 603, medication plan information display and UI controls 604, and a medication purchase options display 605. In an example HAS, the client device provides the medication purchase option screen 600 in response to the user searching a medication name or descriptor in a search box (for example, the search box 601 or the search box 501 of FIG. 5), to the user clicking a medication-related UI control (for example, a medication name in a list of medications in the user's EHR information), etc. The client device may provide the medication purchase option screen 600 during or upon completion of one or more portions of one or more healthcare advisory processes, such as one or more of the processes 202, 203, 207, 209, 211, or other processes. The logic executed by one or more of the client devices or other components in HAS, e.g., the HAS server, to provide the medication purchase option screen 600 is described further with respect to blocks of one or more processes shown in one or more of FIG. 4, 20, or 22.

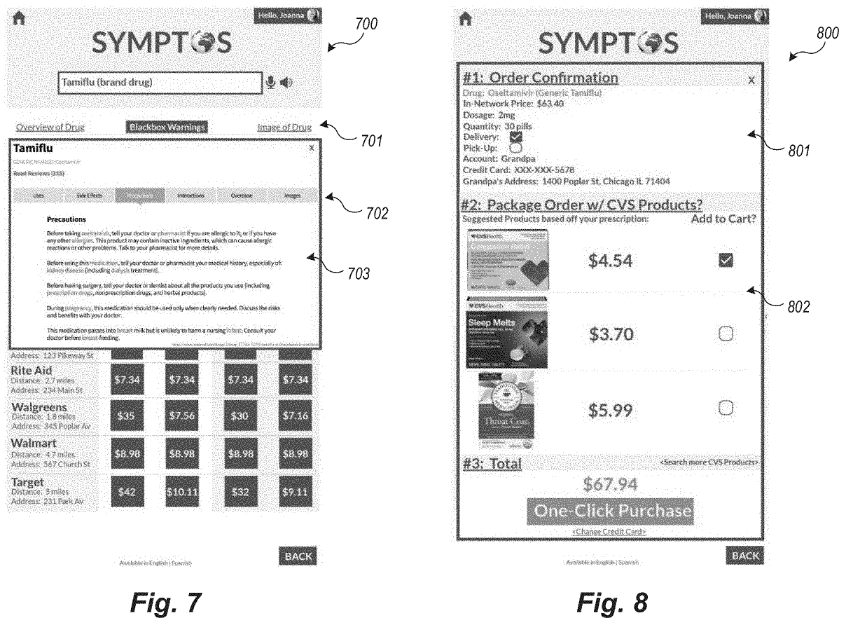

In an example HAS, the search box 601 operates similar to the search box 501 described with reference to FIG. 5. The medication information UI controls 602 include a medication overview UI control, a warnings UI control, and an image of medication UI control. The purchase option location map 603 shows one or more locations associated with the purchase options. The medication purchase option screen 600 may provide the purchase option location map 603 by default. Selecting the medication overview UI control may replace or cover portions of the medication purchase option screen 600, e.g., the map 603, with a window that provides an overview description of the searched medication (see, e.g., similar warnings window in FIG. 7).

The medication plan information display and UI controls 604 may include monetary information related to one or more medication plans or accounts that are relevant to a potential purchase of the searched medication. The monetary information (e.g., expenditure information or medication plan deductible or premium information) may be obtained from the user or one or more insurance provider systems, such as insurance provider systems 113. As shown in FIG. 6, the medication plan information display and UI controls 604 includes a deductible scale (top left), a projection scale (bottom left), a flexible spending account (“FSA”) balance amount (top right), and a health savings account (“HSA”) balance amount (bottom right). These scales are displayed as line scales; however, other representations could be incorporated. For example, the deductible scale shows deductible thresholds for one or more medication plans and shows actual expenditure amounts that are compared to the deductible thresholds under the medication plans. As illustrated, the rightmost dot on the deductible scale represents a deductible threshold for the user's medication plans, the leftmost dot represents the actual amount spent as an individual that counts toward the deductible threshold, and the middle dot represents the actual amount spent as a family that counts toward the deductible threshold.

In an example HAS, the projection scale of UI control 604 shows deductible thresholds for one or more medication plans and shows expenditure amounts as compared to the deductible thresholds under the medication plans. As illustrated, the rightmost dot on the deductible scale represents a deductible threshold for the user's medication plans, the leftmost dot represents an amount predicted to be spent as an individual toward the deductible in a determined timeframe, and the middle dot represents an amount predicted to be spent as a family toward the deductible threshold in the timeframe. One or more components of the HAS, e.g., the HAS server, can predict the expenditure amounts based on historical information associated with the user or other users also covered by the same or similar medication plans. For example, the prediction may be based on generated historical expenditure amounts and forecasts. The flexible spending account (“FSA”) balance amount represents the user's present balance in the user's FSA. The health savings account (“HSA”) balance amount represents the user's present balance in the user's HSA. The HSA balance amount is shown as a scale with the rightmost dot representing the maximum account balance permissible and with the leftmost dot representing the present balance in the user's HSA. In some examples, a rules based analysis is applied to the monetary information to determine which available medication plan is most cost effective for the user based on the user's monetary information and other historical information and the determined medication plan is recommended to the user (e.g., recommending supplemental plans for Medicare patients). A rules based analysis is also applied to the monetary information and other historical information to determine HSA and FSA levels that are most cost effective for the user, and the determined levels are recommended to the user.

The medication purchase options display 605 shows one or more locations where the searched medication is available for purchase and real-time price information for the medication as offered at each of the one or more locations. The price information for each location may include an in-network price and an out-of-network price for a brand-name version of the medication and an in-network price and an out-of-network price for a generic version of the medication. As illustrated, the medication purchase options display includes a brand price in-network column 606, a brand price out-of-network column 607, a generic price in-network column 608, and a generic price out-of-network column 609. The user may select one or more of the price UI controls, e.g., the displayed prices, to initiate or facilitate ordering the medication from the corresponding location.

FIG. 7 is a warnings screen of an example user interface of an example healthcare advisory system. An example warnings screen 700 includes medication information UI controls 701 and a medication information window 702. The medication information UI controls 701 operate similar to the medication information UI controls 602 described with respect to FIG. 6. In some examples, the client device provides medication information window 702 responsive to the user selecting one or more of medication information UI controls 701. For example, the medication information window 702 shows precaution information 703 as a result of selecting “Blackbox Warnings” in the UI controls 701. A user can select one or more tabs in the medication information window 702 to cause the client device to show different information in the medication information window 702. The example of FIG. 7 includes a uses tab, a side effects tab, a precautions tab, an instructions tab, an overdose tab, and an images tab. When selected by the user, each of those tabs may cause the client device to show the corresponding information in the medication information window 702. The window 702 may show medication information (e.g., one or more names of the medication, typical symptoms or illnesses for which the medication is used to treat, a high-level description of how the medication is believed to work, typical forms in which the medication is able to be purchased by patients, recommended doses of the medication, or typical dosage frequencies) as a result of selecting “Overview of Drug” in the UI controls 701.

FIG. 8 is an example order screen of an example user interface of an example healthcare advisory system. The client device may present an example order screen 800 in response to the user selecting a purchase option, e.g., one of the purchase options shown in the medication purchase options display 605 in FIG. 6. FIG. 8 may be provided as part of process 214 in FIG. 2 or initiated by the user selecting a purchase UI control (e.g., the one-click purchase UI control shown at the bottom of the order screen 800). As illustrated, the order screen 800 includes an order confirmation window 801 and a suggested products window 802. The order confirmation window 801 may show order information. For example, the order confirmation window 801 may show the name of the medication associated with the selected purchase option, the price associated with the selected purchase option, the prescribed dosage, the prescribed medication quantity or amount, a user-selected option for the medication to be delivered or picked up at the selected location, a patient for whom the medication is prescribed, information associated with a payment method, and the patient's address.

The suggested products window 802 may show product information associated with products frequently purchased with the medication to provide the user with an opportunity to purchase suggested products with the medication. For example, one or more components of the HAS, e.g., the HAS server, may generate a list of suggested products based on historical purchases that are made along with a purchase of the medication and application or execution of one or more rules against the historical purchase information. The list may include a designated quantity of the most frequently purchased products with the medication, such as the top three most purchased items by users who are purchasing the medication. The list may also be generated based on one or more outputs from one or more artificial intelligence (“AI”) algorithms, e.g., one or more machine learning (“ML”) algorithms, or other methods that the one or more components of the HAS employ with historical purchase information provided by one or more other components in the HAS, e.g., the client devices.

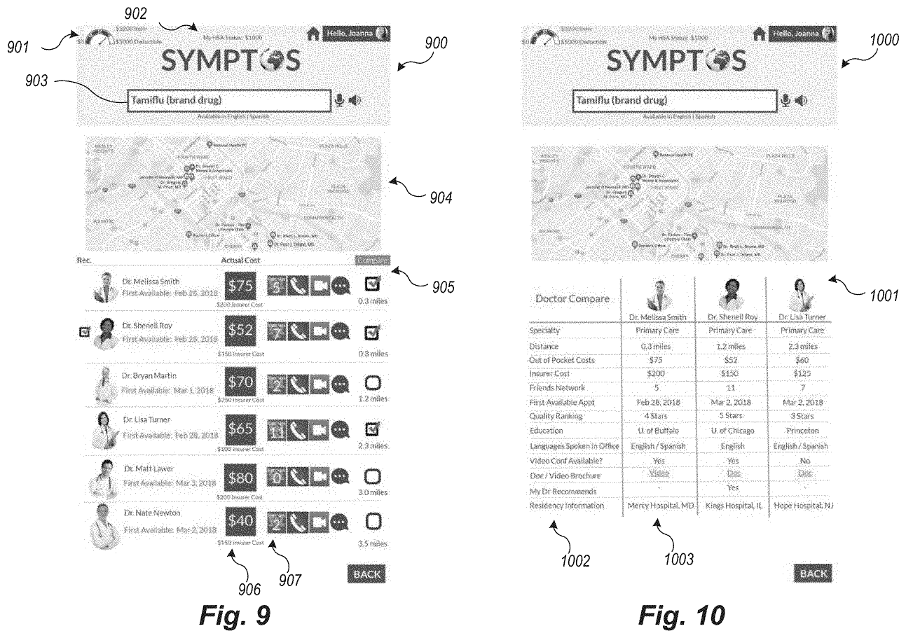

FIG. 9 is a medical provider results screen of an example user interface of an example healthcare advisory system. The example shown in FIG. 9 relates to medical providers that include humans, such as doctors. An example medical provider results screen 900 may be provided as a result of executing one or more portions of process 219 in FIG. 2 or process 2500 in FIG. 25. The medical provider results screen 900 includes a deductible gauge 901, a health savings account (“HSA”) balance amount 902, a search box 903, a medical provider location map 904, and a medical provider recommendations display 905. The medical provider recommendations display 905 may include a UI indicator (e.g., a checkbox and checkmark to the left of one of the recommended medical providers) that indicates which medical provider the HAS has determined is a best match for the user. The deductible gauge 901 and HAS balance amount 902 may operate as described with respect FIG. 6. As with the elements in the medication plan information display and UI controls 604 in FIG. 6, selecting the deductible gauge 901 or the HSA balance amount 902 may cause the client device to display insurance information. For example, selecting one of these components may cause the client device to operate as described with respect to the insurance UI in the “favorite-feature” UI controls 503 in FIG. 5. The search box 903 may operate as described with respect to the search box 501.

In some HAS examples, searching for a type of medical provider (e.g., primary care physicians, vascular surgeons, emergency rooms, or urgent care clinics) causes the HAS to search for one or more medical providers of that type within a designated distance of the user's location ora determined location, to generate a list of one or more recommended medical providers, and to cause the client device to display the one or more recommendations. These actions may be performed in response to user searching based on symptoms, illnesses, or other factors. The medical provider location map 904 may show one or more locations of one or more of the recommended medical providers.

The medical provider recommendations display 905 shows the recommended medical providers with one or more portions of medical provider information associated with each of the recommended medical providers. For example, the medical provider information may include the name of the medical provider, the date of the next available appointment for the medical provider, the cost to the user after insurance payments for a visit to the medical provider, the cost to the user's insurer for a visit to the medical provider, a quantity of the user's friends who visit or have visited the medical provider, a UI control that initiates a telephone call to the medical provider, a UI control that initiates a video conference with the medical provider, a UI control that initiates a text chat communication with the medical provider, a distance between the user or a user selected location and the medical provider, and a UI control to select the medical provider for comparison against one or more other selected medical providers. In some examples, the date or time of the next available appointment may be determined based on rules based calendar synchronization analytics to determine the first time and date that are available on both the user's calendar and the medical provider's calendar. As illustrated, the medical provider recommendations display 905 includes a cost column 906 and a friends column 907. The cost column 906 may include for each recommended medical provider the cost to the user after insurance payments for a visit to the medical provider and the cost to the user's insurer for a visit to the medical provider. The friends column 907 may include for each recommended medical provider a quantity of the user's friends who visit or have visited the medical provider. Also, the HAS may facilitate users connecting with each as a social media platform or through another social media platform and may track which medical provider each of those users visited to generate the information in the friends column 907.

FIG. 10 is a medical provider comparison screen of an example user interface of an example healthcare advisory system. The example illustrated relates to medical providers that include humans, such as doctors. A medical provider comparison screen 1000 may be provided as a result of the user selecting two or more recommended medical providers for comparison in the medical provider results screen 900. The medical provider comparison screen 1000 may include medical provider comparison information window 1001, with a category column 1002 and, for each compared medical provider, a comparison information column 1003, which compares corresponding information for one or more compared medical providers. Compared information may include, for example, specialties of the medical provider, distance between the user or a user selected location and the medical provider, out of pocket costs for the user to visit the medical provider, costs to the user's insurance for the user to visit the medical provider, the quantity of the user's friends who visit or have visited the medical provider, the next available appointment for the user to make with the medical provider, the quality rating of the medical provider as given by other users, the school where the medical provider was educated, one or more languages spoken by the medical provider or staff of the medical provider, whether video conferencing is available with the medical provider, whether the HAS has a brochure document or video associated with the medical provider available for the user's review, whether one or more of the user's present medical providers recommend the compared medical provider, and where the medical provider performed the medical provider's residency.

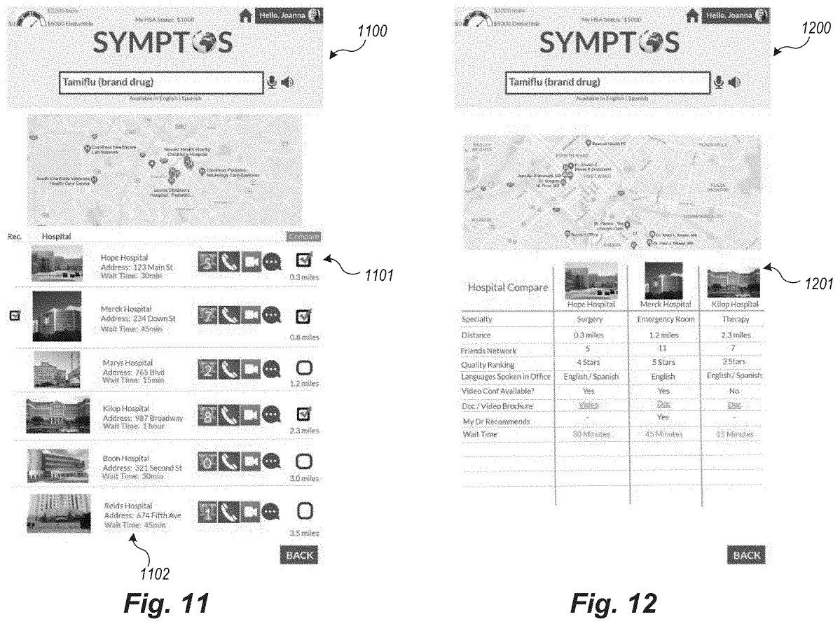

FIG. 11 is a medical provider results screen of an example user interface of an example healthcare advisory system. The example illustrated relates to medical providers that include healthcare facilities, such as hospitals. An example medical provider results screen 1100 is similar to the medical provider results screen 900 and also may be provided as a result of executing one or more portions of process 219 in FIG. 2 or process 2500 in FIG. 25. The medical provider results screen 1100 may include a medical provider recommendations display 1101, which operates in a similar manner as described with respect to the medical provider recommendations display 905. For example, the medical provider recommendations display 1101 may show the present wait time for walk-in appointments at each recommended medical provider. Based upon monitoring the user's client device locations, the HAS can determine when a user has entered or exited a waiting room of a medical provider to generate real-time wait times. The HAS may monitor the user's client device locations over time with location processing engine 307. In other HAS examples, the HAS provides average or computed wait times.

FIG. 12 is a medical provider comparison screen of an example user interface of an example healthcare advisory system. The example shown in FIG. 12 relates to medical providers that include healthcare facilities, such as hospitals. An example medical provider comparison screen 1200 is similar to the medical provider comparison screen 1000 and may be provided as a result of the user selecting two or more recommended medical providers for comparison in the medical provider results screen 1100. The medical provider comparison screen 1200 may include a medical provider comparison information window 1201, which operates in a manner similar to the medical provider comparison information window 1001. For example, the medical provider comparison information window 1201 may show the present wait time for walk-in appointments at each recommended medical provider. The wait times may be generated by the HAS as discussed with respect to FIG. 11.

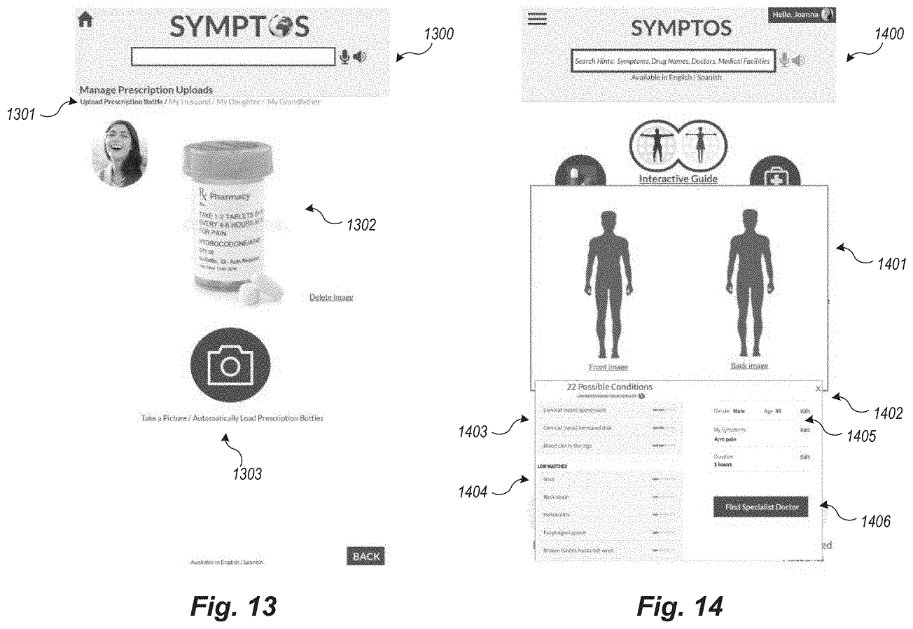

FIG. 13 is a prescription image upload screen of an example user interface of an example healthcare advisory system. An example prescription image upload screen 1300 may be provided during or prior to executing process 203 or one or more blocks shown in FIG. 20 or other figures. For example, the prescription image upload screen 1300 may include an account selector UI control 1301, a medication image display 1302, and an image capture initiation UI control 1303. The client device may transition from one patient's account to another responsive to the user selecting a different account name in the patient selector UI control 1301. The client device may display an image of one or more of a prescription, medication, or medication container responsive to one or more images of one or more prescriptions, medication labels, or other images being uploaded. For example, the user may select the image capture initiation UI control 1303 to facilitate the use of a camera in the client device to take a picture of one or more prescriptions, medication labels, or other objects. In other examples, the image may be uploaded from one or more remotely located users or repositories, such as a medical provider. One or more components of the HAS, e.g., the HAS server or the client device, may evaluate the uploaded images to perform image recognition (e.g., using optical character recognition (“OCR”)) and to generate prescription information based on the evaluated image. The client device may obtain the generated prescription information and may provide the medication image display 1302 based on the generated prescription information.

FIG. 14 is a symptoms input screen of an example user interface of an example healthcare advisory system. An example symptoms input screen 1400 may be provided during or prior to executing the process 217 or one or more blocks shown in FIG. 24 or other figures. The symptoms input screen 1400 may include one or more symptoms input windows 1401 and one or more candidate illness windows 1402. As illustrated, a symptoms input window 1401 includes two graphical representations of a human body: a front representation and a back representation. The user may click, touch, speak, or otherwise select one or more body parts on either or both of the front or rear representations to input a symptom associated with the one or more body parts. Responsive to the user selection, the client device may provide a list of symptoms that the user can select in association with the selected body parts. The client device may prompt the user to input a duration that the user has experienced a selected symptom at a selected body part. The client device may then generate symptoms information based on determined durations, symptoms, and body parts, and provide one or more candidate illnesses in the window 1402.

As illustrated, the candidate illness window 1402 includes a high-ranking matches list 1403, a low-ranking matches list 1404, a pertinent information list 1405, and a find medical provider UI control 1406. One or more components of the HAS, e.g., the HAS server or the client device, evaluates the symptoms information to generate a list of one or more candidate illnesses and may execute or apply one or more rules against the symptoms information to determine a likelihood that the user has each candidate illness. One or more HAS components may then generate a score for each candidate illness based on the likelihood associated with the candidate illness. For example, in FIG. 14, the score for each candidate illness is graphically presented on a scale from one to four, with a score of four representing the highest level of likelihood that the user is experiencing the corresponding candidate illness. The candidate illnesses with the highest scores are included in the high-ranking matches list 1403. The candidate illnesses with the next highest scores are included in the low-ranking matches list 1404. The pertinent information list 1405 includes high-level information used to generate the list of one or more candidate illnesses.

Selecting the find medical provider UI control 1406 causes the HAS to execute code (e.g., the process 219 or the process represented in FIG. 25) to cause the client device to present one or more other screens (e.g., the medical provider results screen 900 or the medical provider results screen 1100). These other screens may be presented based on the highest-ranking candidate illness, the illness category that includes the most candidate illnesses in lists 1403 or 1404, or one or more user- or system-selected candidate illnesses.

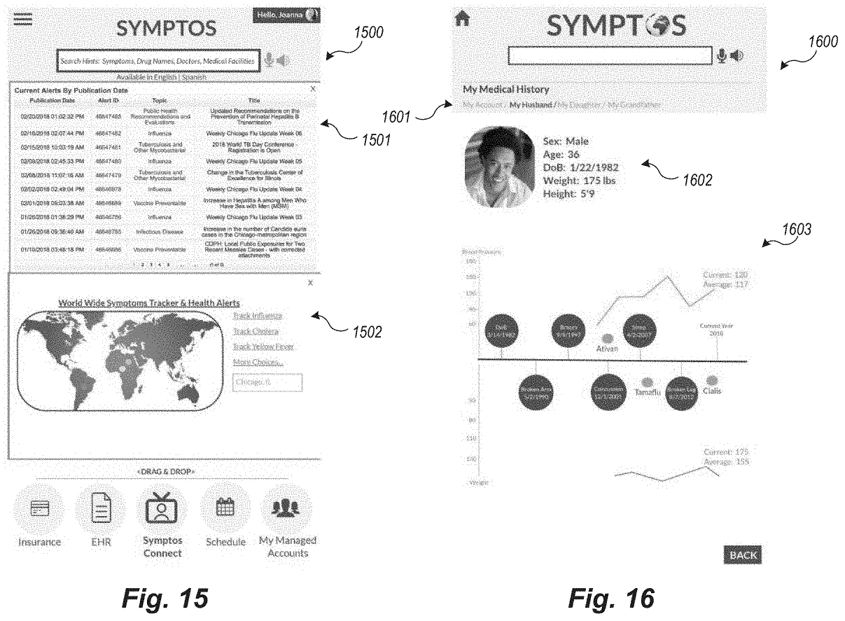

FIG. 15 is a symptoms alert and tracker screen of an example user interface of an example healthcare advisory system. An example symptoms alert and tracker screen 1500 is provided during or prior to executing the process 217 or one or more blocks shown in FIG. 24 or other figures. The client device may provide the symptoms alert and tracker screen 1500 responsive to the user selecting the world health tracker UI control in the HAS process UI controls 502 in FIG. 5. As illustrated, the symptoms alert and tracker screen 1500 includes a current alerts window 1501 and a symptoms tracker window 1502. The current alerts window 1501 displays a list of alert information for multiple health-related alerts. One or more portions of the alert information may be selectable to open the corresponding alert to facilitate the user reading, hearing, or otherwise reviewing the contents of the alert. As illustrated, the health information includes a date and time that an alert was published, an identification number of the alert, a topic of the alert, and a title of the alert. Example topics include public health recommendations and evaluations, influenza, tuberculosis and other mycobacterial, vaccine preventable, and infectious disease. Example titles are illustrated.

The symptoms tracker window 1502 may include a geographical map of one or more portions of the world. The map may be a heat map for one or more symptoms or illnesses that the user or system selects or determines. The map may show the entire world or a geographical region where the user is located, the user selects, or the system determines. Other types of maps and other representations may be similarly incorporated.

In some examples, rules based analytics are applied to the user's medical history information (for example, immunization, symptom, or condition records) and to geolocation information associated with the user's current or impending location to determine whether the user is in or traveling to an area with health alerts that may trigger adverse reactions based on the user's medical history information. Impending location information may be determined based on user selection or predictively based on emails that indicate that the user purchased a plane ticket to the impending location. Calendar analytics may be employed to determine the user's impending travel plans. In some examples, the rules based analytics identify one or more medical provider's in the user's current or impending location that may treat the user's symptoms or conditions or that may treat one or more symptoms or illnesses in the geographical location associated with the health alerts. In other examples, the rules based analytics may identify one or more vaccinations that are available to protect the user from a disease indicated in the notification and, if the logic determines that the user has not taken the vaccination within a preceding time period to render the vaccination still useful, may provide costs and provider locations associated with the vaccinations.

FIG. 16 is a medical history screen of an example user interface of an example healthcare advisory system. An example medical history screen 1600 is provided during or prior to executing one or more blocks shown in FIG. 26 for one or more accounts. The medical history screen 1600 may include account UI controls 1601, a patient information window 1602, and a medical history window 1603. Selecting one or more of the account UI controls 1601 causes the client device to present the medical history screen 1600 with information of an associated account. The patient information window 1602 may include high-level information that describes characteristics of the patient, such as sex, age, date of birth, weight, height, or other information. The medical history window 1603 may show medical history information associated with the patient, such as a graph in which the horizontal axis represents time, and the vertical axis represents a blood pressure scale. The graph may also include, for example, a weight scale or other information tracked over time.

Medical indicators may also be displayed with dates associated with events in the patient's lifetime. For example, the event indicators may include a date of birth indicator, a broken arm indicator, a braces indicator, a concussion indicator, a streptococcal pharyngitis indicator, and a broken leg indicator. Also, the medical indicators may be associated with medications that the user has been prescribed or consumed. For example, the medication indicators may include an ATIVAN indicator, a TAMAFLU indicator, and a CIALIS indicator. The medication indicators for a given medication may be displayed at each date that the patient was prescribed the given medication. Although only blood pressure and weight graphs are displayed, other measurable health values may additionally or alternatively be provided.

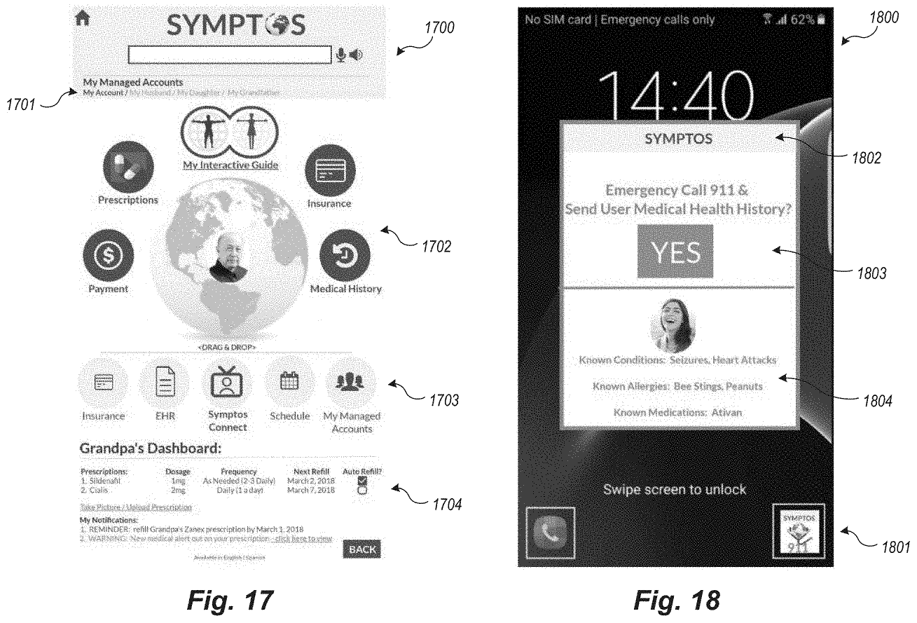

FIG. 17 is an account management screen of an example user interface of an example healthcare advisory system. An example account management screen 1700 is similar to the home screen 500 and may be provided as the home screen when an account that is associated with a patient other than the current user is selected. The management screen 1700 operates in a similar manner as described with respect to the home screen 500 and may include account UI controls 1701, HAS process UI controls 1702, “favorite-feature” UI controls 1703, and a dashboard window 1704. Selecting one or more of the account UI controls 1701 causes the client device to change the account associated with the displayed account management screen. The HAS process UI controls 1702 are selectable by the user to initiate one or more HAS processes, as described similar to FIG. 5. Selecting the interactive guide UI control also initiates one or more HAS processes, as described similar to FIG. 5. The “favorite-feature” UI controls 1703 are rearrangeable or interchangeable by the user and include the same or similar controls as discussed with respect to “favorite-feature” UI controls 503.

The dashboard window 1704 includes prescription information and notification information associated with each of the presently selected patient's current medications. The prescription information may include the medication name, dosage, consumption frequency, and scheduled or projected date of the next refill and may include one or more binary UI controls for each medication to activate or deactivate automatic refills. The notification information may include information associated with reminders or alerts associated with the presently selected patient's medications, conditions, or appointments. For example, the notification information may include a reminder that one of the medications for the presently selected patient is expected to be refilled on an upcoming date. Also, the notification information may include a warning that a new medical alert is available for one of the medications for the presently selected patient, and the alert includes a UI control that, when selected by the user, causes the client device to provide the alert to the user. Other examples of notification information include a notification that a new treatment has been approved by the FDA or another regulating body.

FIG. 18 is an emergency screen of an example user interface of an example healthcare advisory system. When the client device is in a screen-locked state (e.g., after powering on the client device or turning on the screen), the client device may display an example locked screen 1800 and may enable a reduced number of features. Features available in the screen-locked state may include one or more of answering incoming calls, rejecting incoming calls, powering the client device off, unlocking the client device, and making emergency calls. The healthcare advisory application executing on the client device may cause the client device to expand the features available in the screen-locked state when an emergency UI control 1801 is selected. The client device responds by providing an example emergency screen 1802, which includes an emergency action UI control 1803 and an emergency medical information window 1804. Selecting the emergency action UI control 1803 may cause the client device to call an emergency phone number, e.g., 9-1-1, and to provide one or more portions of the user's medical history to emergency personnel. For example, the medical history may be transmitted electronically (for example, email, website submission, etc.), transmitted audibly (for example, automated recording transmitted from the client device during the emergency telephone call), provided visually (for example, displayed in screen-locked screen 1800 for first responders to view when they arrive), by other mechanisms (e.g., local audio through a speaker of the client device). The emergency medical information window 1804 may display information that is likely to be helpful to medical professionals in an emergency. For example, the information may include known conditions of seizures and heart attacks, known allergies to bee stings and peanuts, known medications, emergency contacts, and special instructions customized by the user. The information to display may be determined by executing or applying one or more rules against the user's medical history or may be predetermined information (e.g., predetermined by the user or HAS).

Example embodiments described herein provide applications, tools, data structures and other support to implement a Healthcare Advisory System to be used for facilitating providing users with real-time medication prices. Other embodiments of the described techniques may be used for other purposes, including for optimizing medication purchases, avoiding medication conflicts, and optimizing medical provider selections. In the following description, numerous specific details are set forth, such as data formats and code sequences, etc., in order to provide a thorough understanding of the described techniques. The embodiments described also can be practiced without some of the specific details described herein, or with other specific details, such as changes with respect to the ordering of the logic, different logic, etc. Thus, the scope of the techniques or functions described are not limited by the particular order, selection, or decomposition of aspects described with reference to any particular routine, module, component, and the like.

Also, although certain terms are used primarily herein, other terms could be used interchangeably to yield equivalent embodiments and examples. For example, the phrase “one or more portions of” is sometimes employed in this disclosure with the object of the preposition being a certain type of information to increase readability or to emphasize that less than an entirety of the information may be used, analyzed, or the like. The absence of that phrase in association with a different type of information or in association with a different context does not imply that the entirety of the information is the only intended option.

Further, embodiments described in this disclosure may be described using examples where users or patients have insurance plans and where those users obtain an in-network prescription benefit, yet the embodiments in this disclosure are not so limited. Instead, each embodiment also applies to users without insurance plans. For example, users without insurance plans may also use the HAS to search for medication purchase options from medication supply chain intermediaries, e.g., discount drug providers (“DDPs”), and to obtain real-time price information associated with those medication purchase options.

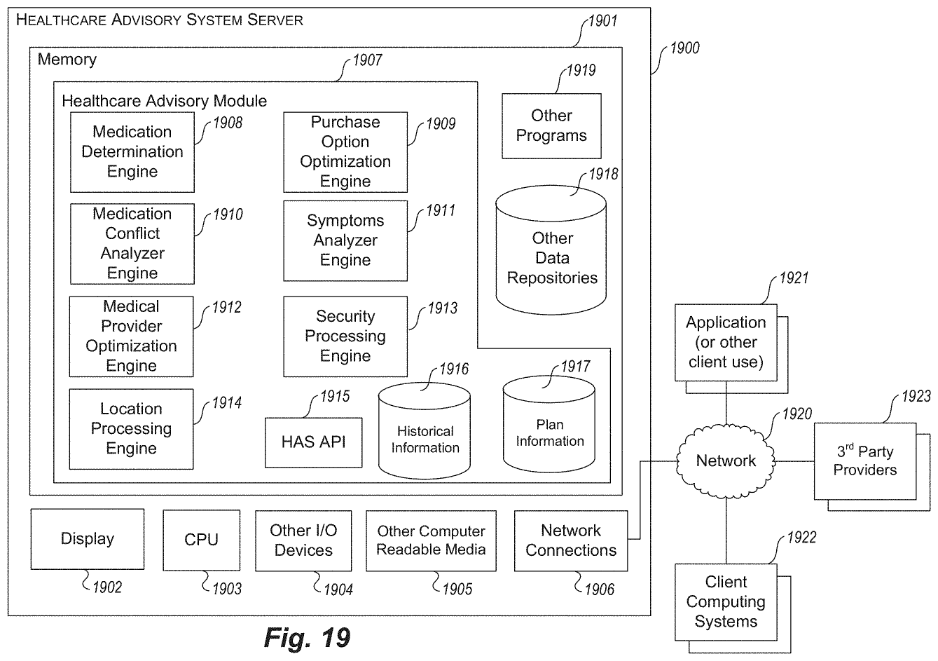

FIG. 19 is an example block diagram of an example healthcare advisory system server of an example healthcare advisory system. Note that one or more general purpose virtual or physical computing systems suitably instructed or a special purpose computing system may be used to implement a HAS server. However, just because it is possible to implement the HAS server on a general-purpose computing system does not mean that the techniques themselves or the operations required to implement the techniques are conventional or well known. Further, the HAS server may be implemented in software, hardware, firmware, or in some combination to achieve the capabilities described herein.

An example HAS server 1900 may comprise one or more server and/or client computing systems and may span distributed locations. In addition, each block shown may represent one or more such blocks as appropriate to a specific embodiment or may be combined with other blocks. Moreover, the various blocks of the HAS server 1900 may physically reside on one or more machines, which use standard (e.g., TCP/IP) or proprietary interprocess communication mechanisms to communicate with each other.

In the embodiment shown, the HAS server 1900 comprises a computer memory (“memory”) 1901, a display 1902, one or more Central Processing Units (“CPU”) 1903, Input/Output devices 1904 (e.g., keyboard, mouse, CRT or LCD display, etc.), other computer-readable media 1905, and one or more network connections 1906. An example healthcare advisory module (“HAM”) 1907 is shown residing in a memory 1901. In other embodiments, some portion of the contents, some of, or all of the components of the HAM 1907 may be stored on and/or transmitted over the other computer-readable media 1905. The components of the HAM 1907 preferably execute on one or more CPUs 1903 and manage the healthcare advisory processes, as described herein. Other code or programs 1919 and potentially other data repositories, such as a data repository 1918, also reside in the memory 1901, and preferably execute on one or more CPUs 1903. Of note, one or more of the components in FIG. 19 may not be present in any specific implementation. For example, some embodiments may not provide means for user input or display.

In example embodiments, the HAM 1907 includes one or more medication determination engines 1908, purchase option optimization engines 1909, medication conflict analyzer engines 1910, symptoms analyzer engines 1911, medical provider optimization engines 1912, security processing engines 1913, and location processing engines 1914. In some examples, the HAM 1907 also includes a HAS application programming interface (“API”) 1915, a historical information repository 1916, and a plan information repository 1917. The historical information repository 1916 may contain historical medical information associated with one or more users. The plan information repository 1917 may contain medical or medication insurance information associated with one or more users. In at least some HAS examples, one or more of the symptoms analyzer engine 1911, the security processing engine 1913, or the location processing engine 1914 is provided external to the HAM 1907 and is available, potentially, over one or more networks 1920. Other and/or different modules may be implemented. In addition, the HAM 1907 may interact via a network 1920 with application or client code 1921 that, for example, uses results computed by the HAM 1907, one or more client computing systems 1922, and/or one or more third-party information provider systems 1923, such as one or more discount drug provider (“DDP”) systems, Federal Drug Administration (“FDA”) systems, pharmacy benefits manager (“PBM”) systems, emergency systems, pharmacy systems, medical provider systems, third-party website systems, or virtual assistant systems. Also, of note, the data repository 1918 may be provided external to the HAM 1907 as well, for example in a knowledge base accessible over one or more networks 1920.

In an example embodiment, components/modules of the HAM 1907 are implemented using standard programming techniques. For example, the HAM 1907 may be implemented as a “native” executable running on the CPU 1903, along with one or more static or dynamic libraries. In other embodiments, the HAM 1907 may be implemented as instructions processed by a virtual machine. A range of programming languages known in the art may be employed for implementing such example embodiments, including representative implementations of various programming language paradigms, including but not limited to, object-oriented, functional, procedural, scripting, and declarative.

The embodiments described above may also use well-known or proprietary, synchronous or asynchronous client-server computing techniques. Also, the various components may be implemented using more monolithic programming techniques, for example, as an executable running on a single CPU computer system, or alternatively decomposed using a variety of structuring techniques known in the art, including but not limited to, multiprogramming, multithreading, client-server, or peer-to-peer, running on one or more computer systems each having one or more CPUs. Some embodiments may execute concurrently and asynchronously and communicate using message passing techniques. Equivalent synchronous embodiments are also supported. Also, other functions could be implemented and/or performed by each component/module, and in different orders, and in different components/modules, yet still achieve the described functions.

In addition, the programming interfaces 1915 to the data stored as part of the

HAM 1907 (e.g., in the data repositories 1916 and 1917) can be available by standard mechanisms such as through C, C++, C#, and Java application programming interfaces (“APIs”); libraries for accessing files, databases, or other data repositories; through scripting languages such as XML; or through Web servers, FTP servers, or other types of servers providing access to stored data. The data repositories 1916 and 1917 may be implemented as one or more database systems, file systems, or any other technique for storing such information, or any combination of the above, including implementations using distributed computing techniques.

Also the example HAM 1907 may be implemented in a distributed environment comprising multiple, even heterogeneous, computer systems and networks. Different configurations and locations of programs and data are contemplated for use with techniques of described herein. In addition, the HAM 1907 may be physical or virtual computing systems and may reside on the same physical system. Also, one or more of the modules may themselves be distributed, pooled or otherwise grouped, such as for load balancing, reliability, or security reasons. A variety of distributed computing techniques are appropriate for implementing the components of the illustrated embodiments in a distributed manner including but not limited to TCP/IP sockets, RPC, RMI, HTTP, Web Services (XML-RPC, JAX-RPC, SOAP, etc.), web sockets, and the like. Other variations are possible. Also, other functionality could be provided by each component/module, or existing functionality could be distributed amongst the components/modules in different ways, yet still achieve the functions of a HAM.

Furthermore, in some embodiments, some or all of the components of the HAM 1907 may be implemented or provided in other manners, such as at least partially in firmware and/or hardware, including, but not limited to one or more application-specific integrated circuits (ASICs), standard integrated circuits, controllers executing appropriate instructions, and including microcontrollers and/or embedded controllers, field-programmable gate arrays (FPGAs), complex programmable logic devices (CPLDs), and the like. Some or all of the system components and/or data structures may also be stored as contents (e.g., as executable or other machine-readable software instructions or structured data) on a computer-readable medium (e.g., a hard disk; memory; network; other computer-readable medium; or other portable media article to be read by an appropriate drive or via an appropriate connection, such as a DVD or flash memory device) to enable the computer-readable medium to execute or otherwise use or provide the contents to perform at least some of the described techniques. Some or all of the components and/or data structures may be stored on tangible, non-transitory storage mediums. Some or all of the system components and data structures may also be stored as data signals (e.g., by being encoded as part of a carrier wave or included as part of an analog or digital propagated signal) on a variety of computer-readable transmission mediums, which are then transmitted, including across wireless-based and wired/cable-based mediums, and may take a variety of forms (e.g., as part of a single or multiplexed analog signal, or as multiple discrete digital packets or frames). Such computer program products may also take other forms in other embodiments. Accordingly, embodiments of this disclosure may be practiced with other computer system configurations.

FIGS. 20 and 22-26 illustrate example logic for the components of a HAS as described with respect to FIGS. 1-19.

FIG. 20 is an example flow diagram of real-time pricing advisory logic performed by an example healthcare advisory server. For example, real-time pricing advisory logic 2000 may be performed by one or more of the medication determination engine 301 of FIG. 3, the purchase option optimization option engine 302 of FIG. 3, the security processing engine 306 of FIG. 3, the location processing engine 307 of FIG. 3, the medication determination engine 1908 of FIG. 19, the purchase option optimization option engine 1909 of FIG. 19, the security processing engine 1913 of FIG. 19, or the location processing engine 1914 of FIG. 19. The logic 2000 is responsible for providing real-time medication pricing advice.

Specifically, in block 2001, the logic obtains an indication of medication that a user intends to purchase, for example, from one or more medication determination engines. The indication may be based on one or more photographs of one or more prescriptions, medication labels, or doses of the medication or based on one or more user inputs or medical provider inputs. The indication may also be generated or otherwise received. The medication indicator may include the name of the medication, the prescribed dosage, the prescribed dosage interval, and the prescribed quantity or amount of the medication.

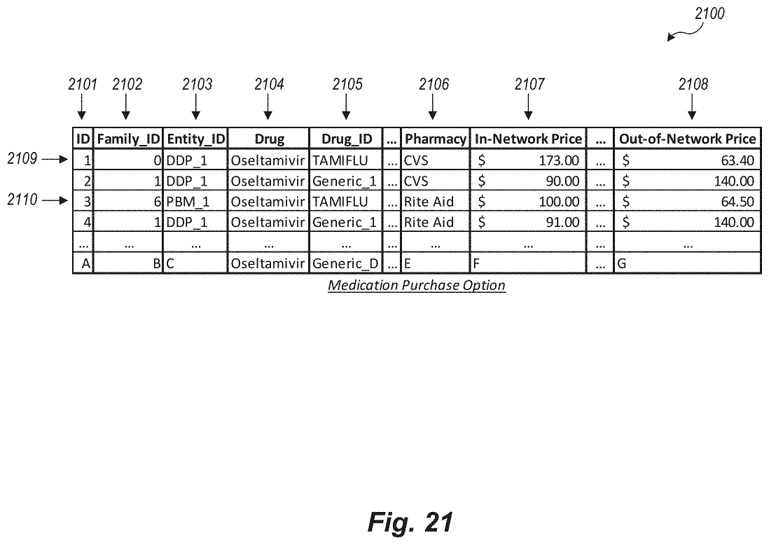

In block 2002, the logic accesses one or more discount drug provider (“DDP”) application programming interfaces (“APIs”) to obtain medication price information associated with the indicated medication from one or more DDP systems. The one or more DDP APIs facilitate communicating with the one or more DDP systems to retrieve or receive medication price information associated with the indicated medication from the DDP system(s). The medication price information may include in-network and out-of-network prices of name-brand and generic versions of the indicated medication at one or more pharmacies. The logic may cause generation of one or more medication price information data objects based on the obtained medication price information, which are discussed further with respect to FIG. 21. The one or more pharmacies may include pharmacies within a designated territory, e.g., a state, a county, a city, a postal code, or a neighborhood, or within a designated range of a given location. The location may be determined based on geolocation information obtained from one or more components in a client device associated with the user, e.g., a GPS receiver in the client device or one or more designated locations.

In block 2003, the logic optionally notifies one or more pharmacy benefits managers (“PBMs”) that the user will potentially deviate from the user's medication plan. For example, the logic may determine that the lowest price for the indicated medication is associated with a purchase option that is out-of-network with respect to the user's medication plan. Block 2003 may be optional because, in a given instance, the logic may determine that the user is unlikely to deviate from the user's medication plan or the one or more PBMs and, thus, does not notify the one or more PBMs of the determined potential deviation.

The real-time pricing advisory logic determines whether the user will potentially deviate from a medical plan or PBM by evaluating the obtained medication price information based upon rules executed or applied against the obtained medication price information. For example, the rules may compare the medication prices indicated by the obtained medication price information, and, based upon conditions matching or otherwise satisfying one or more of the rules, determine that the user is expected to deviate from the user's medication plan and the one or more PBMs. Conditions satisfying the rules include, for example, discovering that the lowest medication price is associated with a purchase option that is outside of the medication plan and the one or more PBMs, discovering that the pharmacy that is closest to the user's location or the user selected location is associated with a purchase option that is outside of the medication plan and the one or more PBMs, determining that the lowest-priced purchase option is within a user- or system-determined distance (e.g., the logic may dynamically vary the distance based on the price difference between the lowest-priced purchase option and the lowest-priced in-network option) of the user's location or designated location, or other based upon other factors. In some cases, the logic executes one or more Al algorithms to learn which rules result in a user purchasing outside a medication plan, such as a discount drug provider (“DDP”). In other cases, the logic executes one or more predefined scripts or configuration files that define the one or more rules (stored, for example, in a repository).

In block 2004, the logic determines whether the one or more DDPs (e.g., one or more pharmacy benefits managers (“PBMs”)) has opted to revise one or more prices in response to the one or more DDPs being notified of the user's potential deviation and, if so, continues to block 2005, otherwise continues to block 2006. For example, a DDP may reduce a price to the user for one or more purchase options to incentivize the user to select a purchase option that is in the user's medication plan and the DDP.

In block 2005, the logic revises the medication price information associated with the purchase options based on the reduced prices received from the PBMs. The logic may revise the medication price information by modifying one or more medication price information data models. The logic then continues to block 2006. Medication price information data models are discussed further with respect to FIG. 21.

In block 2006, the logic optionally accesses a pharmacy application programming interface (“API”) to order medication. Block 2006 may be optional because, in a given instance, the user may opt to not fill a prescription for the medication. The pharmacy API facilitates the logic communicating with a pharmacy system of a pharmacy that is associated with a selected purchase option. The logic may provide the one or more purchase options to the user and, responsive to user selection, search (e.g., look, examine, etc.) order instructions (including an identification of a pharmacy API) for the pharmacy that is associated with a selected purchase option. The logic may then execute the order instructions to order the medication. The logic then ends.

FIG. 21 is an example medication price information data model generated by an example healthcare advisory server. An example medication price information data model 2100 may include one or more data objects (for example, records or other objects) that represent one or more medication purchase options used by the HAS logic, for example, of FIG. 20. The data model 2100 may be implemented using any type of data structure that supports multidimensional information such as database records, an array, a linked list, and the like. The data model 2100 may include a number of named attributes, such as an ID 2101, a Family_ID 2102, an Entity_ID 2103, a Drug 2104, a Drug_ID 2105, a Pharmacy 2106, an In-Network Price 2107, and an Out-of-Network Price 2108. For each data object, the values of identifiers, such as those shown as entries for the attribute 2101 or other attributes, may be sequential numbers, non-sequential numbers, discrete values, strings, or other values. Each data object may be defined or characterized by one or more values associated with the named attributes. For example, the data object 2109 with an ID 2101 of “1” has an Entity_ID 2103 of “DDP_1”, a Drug 2104 of “Oseltamivir”, a Drug_ID 2105 of “TAMIFLU”, a Pharmacy 2106 of “CVS”. As shown, the data object 2109 represents a medical purchase option that is associated with Oseltamivir under the brand name TAMIFLU being offered at CVS with In-Network Price of $173 and Out-of-Network Price of $63.40. In contrast, a data object 2110 with an ID 2101 of “3” is associated with Oseltamivir under the brand name TAM IFLU being offered at RITE AID with In-Network Price of $100 and Out-of-Network Price of $64.50 and has appropriated associated values.

In some examples, the HAS server logic generates the data model 2100 based on obtained medication price information. For example, the logic may generate a data object to include in the data model 2100 for one or more purchase options associated with the obtained medication price information, populating the data object with values based on the obtained medication price information. As illustrated, a data object is generated for each drug identifier at each pharmacy associated with the purchase options.

In some scenarios, if the data model 2100 involves hierarchies (for example, trees or other data structures), nested data models or objects, or other relationships, values associated with the attribute 2102 may reference identification values associated with related purchase options. Examples of relationships may include being associated with the same or related discount drug providers (“DDPs”), the same or related pharmacy benefits managers (“PBMs”), the same or related medications, the same medication name, the same or related pharmacies, geographical location, taxonomy information, or other information. For clarity, the illustrated portion of the data model 2100 is shown using tabular format. The data sets or data objects also may be arranged differently, such as using different formats, data structures, objects, or in other arrangements.

FIG. 22 is an example flow diagram of price information analysis logic performed by an example healthcare advisory server. One or more portions of example price information analysis logic 2200 may be performed by one or more purchase option optimization engines or security processing engines, such as the purchase option optimization engine 302 of FIG. 3, the security processing engine 306 of FIG. 3, the purchase option optimization engine 1909 of FIG. 19, or the security processing engine 1913 of FIG. 19. The 2200 is responsible for determining a medication purchase option that is predicted to best suit the user.

Specifically, in block 2201, the logic analyzes historical information (e.g., historical pharmaceutical usage or expense information) to predict the medical expenses (e.g., pharmaceutical expenses) associated with the user for a remainder of a determined timeframe, for example, based on historical information to exclude or reduce a weight applied to anomaly expenses from the past medical expenses. The logic may estimate the user's past medical expenses based on average costs for the user's past medical treatments or other medical events that are identified in the historical information. The historical information may include past medical expenses for other users having the same or similar characteristics as the user, e.g., demographics characteristics, location characteristics, illness characteristics, or other characteristics. The logic may extrapolate from the medical expenses through the remainder of the timeframe, and, if one or more impending anomaly expenses are expected to be incurred (e.g., user entered, scheduled in the historical information, or otherwise determined), they may be added to the extrapolated expenses. The logic may provide notification of one or more predicted or estimated values that led to the determination (e.g., expected pharmaceutical expenses for the timeframe) to facilitate the user considering whether the expected expenses were predicted based on an anomaly in the historical information. The logic also may facilitate the user modifying a portion of the historical information employed in making the prediction.

In block 2202, the logic analyzes medication plan information to predict the user's out-of-pocket medical costs over the remainder of the timeframe, for example, by generating expense threshold information based on the medical plan information (e.g., medication plan information such as the user's pharmacy insurance plan information (for example, deductible amount, premium amount, or other insurance information), flexible spending account (“FSA”) information (for example, current balance or typical or predicted contribution amounts), or health savings account (“HSA”) information (for example, current balance or typical or predicted contribution amounts)). The plan information may include insurance information associated with one or more insurance plans or discount drug providers (“DDPs”). The expense threshold information may indicate one or more discovered expense thresholds associated with one or more of the user's medication plans (e.g., one or more of the user's insurance plans or DDPs), such as one or more deductible thresholds, maximum annual payments by an insurer that provides the medication plan, one or more out-of-pocket maximums, or other amounts. The logic may predict the user's out-of-pocket medical costs for the remainder of the timeframe based on application of the expense threshold information to the predicted and past medical expenses for the timeframe. The logic may identify which portions of the predicted and past medical expenses are applicable to which of the one or more expense thresholds and/or which of the one or more expense thresholds have been or are expected to be met for the timeframe. The logic may determine the portions of the medical expenses that are expected to be paid by the insurance company that provides the medication plan to predict the user's out-of-pocket medical costs based on the portion of the predicted and past medical expenses that is expected to remain unpaid by the insurance company. One or more of the analyzed historical information or the analyzed medication plan information may be reduced to those portions of information that are related to one or more medication purchase options that the user is considering.

In block 2203, the logic determines whether a sufficient amount of information has been analyzed to predict the user's out-of-pocket medical costs and, if so, continues to block 2205, otherwise continues to block 2204. For example, the logic may determine sufficiency of information by generating a confidence score for a predicted out-of-pocket medical cost and, if the confidence score falls below a threshold, assess that further information should be analyzed and the predicted medical cost recalculated to provide a new predicted medical cost having a higher confidence score. If the confidence score falls below the threshold after a designated number of recalculations, the logic may continue to block 2205.

In block 2204, the logic determines and obtains further information to be used in predicting the user's out-of-pocket medical costs over the remainder of the timeframe, for example, by discovering one or more types of historical information that were not included in the block 2203 analysis. For example, the logic may obtain information or types of information that was not previously considered from the user, a repository, or another HAS component. The logic then continues at block 2201 to perform the analysis of the previously analyzed historical information along with the newly obtained information. The logic also may select one or more different rules to execute or apply in the analysis of one or more of blocks 2201 or 2202 to attempt to obtain a higher confidence score.

At block 2205, the logic compares one or more medication purchase options (e.g., one or more purchase options provided by the process 2000) based on the predicted out-of-pocket medical expense amounts, for example, by determining an expected out-of-pocket cost to the user for each of the one or more purchase options over the remainder of the timeframe. The logic may generate predicted out-of-pocket costs for each purchase option alone or for each purchase option along with predicted out-of-pocket costs for the user's predicted medical expenses over the remainder of the timeframe.

At block 2206, the logic determines and provides one or more medication purchase options that are predicted to best suit the user based on one or more pricing prediction rules, for example, based on determining which purchase option is predicted to provide the user with the lowest out-of-pocket costs for the remainder of the timeframe or determining which purchase option has the highest confidence score among multiple purchase options predicted to provide the same or similar out-of-pocket costs. The logic may determine that multiple predicted costs are similar based on the costs being within a designated range of each other. The logic then ends.

FIG. 23 is an example flow diagram of medication analysis logic performed by an example healthcare advisory server. One or more portions of example medication analyzer logic 2300 may be performed by one or more medication determination engines, medication conflict analyzer engines, or security processing engines, such as the medication determination engine 301 of FIG. 3, the medication conflict analyzer engine 303 of FIG. 3, the security processing engine 306 of FIG. 3, the medication determination engine 1908 of FIG. 19, the medication conflict analyzer engine 1910 of FIG. 19, or the security processing engine 1913 of FIG. 19. The logic 2300 is responsible for generating one or more alerts if one or more analyzed medications are likely to contribute to the user experiencing one or more adverse effects.

Specifically, in block 2301, the logic compares medication information to historical medical information. The logic may analyze the medication information to discover one or more medication conflicts associated with the medication identified by the medication information, e.g., one or more adverse medication interactions with one or more other medications or classes of medications. The logic may evaluate the historical medical information to determine whether the user presently uses the one or more discovered medications or one or more medications in the one or more discovered medication classes.

In block 2302, the logic determines whether the user is particularly susceptible to the identified medication conflicts and, if so, continues to block 2304, otherwise continues to block 2303. The logic may analyze the comparison results of block 2301 to determine a likelihood or severity level for each expected medication conflict. The logic may determine that a medication conflict is likely to present itself if a corresponding likelihood or severity level exceeds one or more determined thresholds. For example, the logic may apply a lower likelihood threshold for medication conflicts associated with greater severity levels than medication conflicts associated with lower severity levels. Severity levels may be determined relative to each other or one or more user- or system-determined thresholds.

In block 2303, the logic optionally updates one or more portions of the historical information based on the comparison results of block 2301. Block 2303 is optional because the logic may update the historical information to reflect that the user has been cleared to consume the medication associated with the medication information or may leave the historical information unaltered to force the logic to reevaluate potential conflicts each time the logic evaluates a medication.

In block 2304, the logic generates one or more alerts (e.g., notifications), optionally based on severity or type of expected medication conflict, for example, by generating a higher warning alert if one or more of the likelihood or the severity level for an expected medication conflict is high or a lower warning alert if one or more of the likelihood or the severity level is low (e.g., relative to one or more user- or system-determined thresholds). For example, if the alert is audio based, the tone or volume may correlate to severity level. The logic may provide the one or more alerts to the user, one or more medical providers (e.g., a medical provider that prescribed the analyzed medication), or one or more pharmacies. The one or more alerts may include medication conflict information, for example, including one or more indicators of one or more of the type of the medication conflict, the likelihood of the medication conflict, or the severity of the medication conflict.

In block 2305, the logic optionally determines and provides one or more medication alternatives, for example, by evaluating the medication information and the historical information to determine one or more medications that can be used to treat the same one or more conditions or symptoms that the analyzed or currently used medication was prescribed to treat and that have no, fewer, or less sever expected medication conflicts. Block 2305 is optional because process 2300 may end after providing the one or more alerts. In some examples, the logic may provide the one or more determined alternative medications to the user, one or more medical providers, or one or more pharmacies. The logic then ends.

FIG. 24 is an example flow diagram of symptoms analysis logic performed by an example healthcare advisory server. One or more portions of example symptoms analyze logic 2400 may be performed by one or more symptoms analyzer engines, such as the symptoms analyzer engine 304 of FIG. 3 or the symptoms analyzer engine 1911 of FIG. 19. The logic 2400 is responsible for generating one or more notifications of candidate illnesses based on symptom information.

Specifically, in block 2401, the logic determines and provides symptom information, for example, by obtaining one or more user inputs or by generating the symptom information from sensor information obtained from wearable sensors, implanted sensors, or other sensors. The symptom information may include one or more indicators of one or more body parts associated with the symptoms, one or more descriptors of the type of symptom, one or more indicators of a duration that the user has been experiencing a symptom, and one or more indicators of a severity level of a symptom.

In block 2402, the logic analyzes the symptom information to generate information for one or more candidate illnesses, for example, by selecting one or more candidate illnesses based on searching (e.g., looking, examining, etc.) for illnesses in one or more repositories associated with symptoms that are the same as or similar to the symptoms indicated by the symptoms information.

In block 2403, the logic determines whether a sufficient amount of information has been analyzed to diagnose the user's illness and, if so, continues to block 2405, otherwise continues to block 2404. For example, the logic may determine sufficiency of information by generating one or more confidence scores that indicate a likelihood that the user is suffering from the one or more candidate illnesses. If the one or more confidence scores fall below one or more user- or system-determined thresholds, the logic may determine that further information should be analyzed and new candidate illness information generated to achieve one or more higher confidence scores. If the one or more confidence scores fall below the one or more thresholds after a determined number of attempts, the logic may continue to block 2405.

In block 2404, the logic determines and provides further information to be used in generating candidate illness information, for example, by discovering additional types of symptom information that were not included in the analysis and obtaining (e.g., from one or more sensors, the user, or a medical provider) additional symptom information corresponding to the types not previously considered. The logic then returns to block 2401 to again attempt to generate candidate illness information. The logic also may select one or more different rules to execute or apply in one or more of blocks 2401 or 2402 to attempt to obtain a higher confidence score.

In block 2405, the logic generates one or more notifications of candidate illness information, for example, for a user- or system-determined number of the one or more candidate illnesses based on which candidate illnesses have the highest-ranking confidence scores. The candidate illness information may include the one or more confidence scores. The logic may provide the one or more notifications to the user or one or more medical providers.

In block 2406, the logic optionally executes medical providers analyzer logic (e.g., the logic represented by one or more of the blocks shown in FIG. 25) based on the candidate illness information. Block 2406 is optional because the user may instruct the logic to not send the information to a medical provider. The logic then ends.

FIG. 25 is an example flow diagram of medical providers analysis logic performed by an example healthcare advisory server. The medical providers analyze logic 2500 may be performed by one or more medical provider optimization engines or location processing engines, such as the medical provider optimization engine 305 in FIG. 3, the location processing engine 307 in FIG. 3, the medical provider optimization engine 1912 in FIG. 19, or the location processing engine 1914 in FIG. 19. The logic 2500 is responsible for recommending one or more medical providers based on illness information.

Specifically, in block 2501, the logic determines and provides illness information, for example, including candidate illness information, such as candidate illness information generated by the process 2400 in FIG. 24. The illness information also may be obtained from a user or a medical provider that diagnosed the user with one or more illnesses associated with the illness information.

In block 2502, the logic analyzes the illness information to generate candidate medical provider information, for example, by generating illness category information or medical provider type information and searching one or more repositories for one or more medical providers associated with the illness categories or medical provider types based on the illness category or medical provider type information. The logic may obtain illness category or medical provider type information by searching one or more repositories based on the illness information. The logic may filter the medical providers based on location, such as based on a user- or system-determined distance from the user's location or a determined or designated location.

In block 2503, the logic determines whether a sufficient amount of information has been analyzed to generate the candidate medical provider information and, if so, continues to block 2505, otherwise continues to block 2504. For example, the logic may determine sufficiency of information by generating one or more confidence scores that indicate a likelihood that the medical providers identified by the candidate medical provider information treat one or more illnesses or symptoms identified by the illness information. If the confidence scores fall below one or more thresholds, the logic may determine that more or different information should be analyzed and new candidate medical provider information generated to achieve one or more higher confidence scores. If the confidence scores fall below the one or more thresholds after a user- or system-determined number of attempts, the logic may continue to block 2505.

In block 2504, the logic determines and provides more or different information to be used in generating candidate medical provider information, for example, by discovering one or more types of illness information that were not included in the analysis. The logic may obtain (e.g., from the user or one or more medical providers) information regarding types of illness information not previously considered. The logic then continues at block 2501 to again generate candidate medical provider information. The logic also may select one or more different rules to execute or apply in blocks 2501 or 2502 to obtain a higher confidence score.

In block 2505, the logic generates notifications of candidate medical provider information, for example, for a user- or system-determined number of the candidate medical providers based on which candidate medical providers have the highest-ranking confidence scores. The candidate medical provider information may include the one or more confidence scores. The logic may provide the one or more notifications to the user.

In block 2506, the logic optionally executes logic to generate and provide historical information (e.g., the logic represented by one or more of the blocks shown in FIG. 26) based on the candidate medical provider information. The logic then ends.

FIG. 26 is an example flow diagram of generate and provide historical information logic performed by an example healthcare advisory server. The generate and provide historical information logic 2600 may be performed by one or more security processing engines, such as the security processing engine 306 in FIG. 3 or the security processing engine 1913 in FIG. 19. The logic 2600 is responsible for increasing security of user information when providing historical information associated with the user. For example, the logic may ensure that electronic health record (“EHR”) information to be provided to a target entity complies with one or more requirements or guidelines defined in one or more standards, for example, the Health Insurance Portability and Accountability Act of 1996 (“HIPPA”).

Specifically, in block 2601, the logic determines and provides historical information, for example, by obtaining historical medical information associated with the user from one or more repositories, the user, or one or more medical providers.

In block 2602, the logic optionally analyzes one or more user preferences to generate one or more portions of screened historical information, for example, based on an indication in a user preference that the user prefers that certain categories of the historical information be omitted and not transferred to one or more target entities, e.g., medical providers, pharmacies, discount drug providers (“DDPs”), or pharmacy benefits managers (“PBMs”). Block 2602 is optional because the user may not have provided any user preferences or because the user may not be provided with the ability to modify the user preferences..

In block 2603, the logic analyzes target entity information to generate filtered historical information, for example, by filtering the screened historical information based on one or more indicators in the target entity information that indicate types or categories of historical information that the target entity requests. The target entity information may identify one or more target entities (e.g., a specialist doctor or facility) that the logic intends to provide with versions of the historical information. For example, a specialist medical provider may request historical information in one or more categories that are particularly relevant to the specialty practice area of the specialist medical provider. The logic may obtain the target entity information from the user, the target entity, or another component of the HAS. The logic also may filter the screened historical information based on a determination that one or more portions of the historical information are related to symptoms, illness, or candidate illness information (e.g., same body part, same or similar symptoms, association with time frame or past medical events such as medication, injury, illness, surgery, or other events).

In block 2604, the logic determines whether identity information associated with the user is available to the target entity and, if so, continues to block 2606, otherwise continues to block 2605. The logic may determine that the target entity should not be made aware of the user's identity, for example, based on the target entity including an aggregating data repository that collects historical information to discover rules, patterns, or other techniques or to train algorithms. The logic also may determine that the target entity should be made aware of the user's identity, for example, based on the target entity including a medical provider with which the user has an impending medical appointment. The target identity information may include an indicator that the logic analyzes to determine whether the identity information is available to the target entity. The identity information may include one or more of the user's name, social security number, house address, or other identifying information. In some instances, the target identify information is encoded to comply with requirements such as requirements of a target entity.

In block 2605, the logic anonymizes the filtered historical information, for example, by removing identifying information. The logic also may encrypt the filtered historical information.

In block 2606, the logic optionally provides the filtered or encoded historical information to the one or more target entities. Block 2606 is optional because the logic may provide the filtered historical information to the user, who then provides the filtered historical information to the one or more target entities. The logic then ends.

The HAS server may provide a template to one or more medical provider systems or medical provider client devices to facilitate a medical provider creating digital prescriptions. The HAS may facilitate the medical provider speaking one or more identifiers of the medical provider (e.g., the medical provider's name), obtain the National Provider Identifier (“NPI”) associated with the medical provider, and facilitate the medical provider confirming the obtained NPI. The HAS may facilitate the medical provider speaking one or more portions of prescription information, and populate the digital prescription template based on one or more portions of the spoken prescription information. The HAS may generate one or more portions of medication information, e.g., default dosage information or quantity information, and present the one or more portions of medication information to the medical provider for the medical provider's approval. The HAS may obtain and provide real-time purchase option information associated with the medication identified in the prescription information to facilitate the medical provider and the patient discussing the medication purchase options associated with the purchase option information. The HAS may facilitate the medical provider electronically signing the digital prescription and route the digital prescription to a pharmacy associated with a selected medication purchase option for fulfillment of the prescription.

In some examples, the user may be enabled to activate a speaker feature that facilitates vocal, gesture, or touch user input controls. In response to one or more user commands, information displayed to the user is read out loud to the user. The information implements a set of rules to analyze the displayed information to prioritize the information to improve conciseness of the reading. For example, when the user searches for a medication, the lowest cost price listed in a given row or across all rows will be read out loud first. After one or more portions of information are read out loud to the user, the user is asked whether the user wants further displayed information read out loud. Based on rules analytics, each portion of the displayed information is logically associated with a score that indicates a level of sensitivity of the portion of information (i.e., how private the information is). The user may be prompted with a vocal statement or question to provide an instruction as to whether the current time and location is appropriate to read out loud portions of information having scores that indicate high levels of sensitivity (e.g., medication name or symptom information).

From the foregoing it will be appreciated that, although specific embodiments have been described herein for purposes of illustration, various modifications may be made without deviating from the spirit and scope of the invention. For example, the methods and systems for obtaining real-time medication prices, optimizing medication purchases, avoiding medication conflicts, and optimizing medical provider selections discussed herein are applicable to other architectures other than a client-server architecture. Also, the methods and systems discussed herein are applicable to differing protocols, communication media (optical, wireless, cable, etc.) and devices (such as wireless handsets, electronic organizers, personal digital assistants, portable email machines, game machines, pagers, navigation devices such as GPS receivers, etc.). For the purposes of this disclosure, the term “or” refers to a grammatical conjunction to indicate that one or more of the connected terms may be employed (“and/or”). For example, the phrase “one or more A, B, or C” is employed to discretely disclose each of the following: i) one or more As, ii) one or more Bs, iii) one or more Cs, iv) one or more As and one or more Bs, v) one or more As and one or more Cs, vi) one or more Bs and one or more Cs, and vii) one or more As, one or more Bs, and one or more Cs.

All of the above U.S. patents, U.S. patent application publications, U.S. patent applications, foreign patents, foreign patent applications, non-patent publications, and appendixes referred to in this specification and/or listed in the Application Data Sheet, including but not limited to U.S. Patent Application No. 62/738,992, filed on Sep. 28, 2018 and entitled “HEALTHCARE ECOSYSTEM METHODS, SYSTEMS, AND TECHNIQUES” is incorporated herein by reference, in its entirety.

1. A method in a computing system for improving electronic communication in a healthcare environment comprising a plurality of heterogeneous computing devices operated by distinct parties, comprising:
under control of a first computing device,
receiving an indication of medication that a user intends to purchase;
determining and providing medication information associated with the indicated medication;
accessing a drug supply chain intermediary application programming interface (“API”) of computing instructions executing on a second computing device distinct from the first computing device and operated by a first external third party to obtain medication price information based on the provided medication information, the obtained medication price information including a discount price associated with purchase of the medication from one or more drug supply chain intermediaries and a discount drug provider (“DDP”) medication price associated with a plan that is associated with the user and a DDP system;
generating a medication price information data object based on the provided medication information and the obtained medication price information, the medication price information data object having a plurality of fields that each have one or more values;
in near real-time,
notifying the DDP system through a third computing device distinct from and external to the first computing device that the user is considering purchasing the medication for the discount price from an entity that is outside of the plan associated with the user and the DDP system;
obtaining a revised DDP medication price from the DDP system based on the notification to the DDP system, the revised DDP medication price indicating a more competitive price than the discount price;
automatically generating revised medication price information, by revising one or more values of one or more fields in the medication price information data object, based on the revised DDP medication price; and
presenting medication purchase options to the user based on the generated revised medication price information and the determined target entity;
receiving from the user an indication of a selected medication purchase option from the presented medication purchase options;
accessing a pharmacy API of computing instructions executing on a fourth computing device distinct from the first computing device and operated by a second external third party to provide the medication information to a pharmaceutical entity based on the indicated medication purchase option.

2 The method of claim 1, further comprising:
determining the pharmaceutical entity based upon a past purchase of a same medication.

3 The method of claim 1 wherein determining the medication information associated with the medication that the user intends to purchase comprises generating the medication information based on an image of a prescription or a medication label.

4. The method of claim 3 wherein generating the medication information based on the image of the prescription or the medication label further comprises:
obtaining a set of pre-processing rules;
obtaining a set of word- or character-recognition rules;
capturing an image of the prescription or the medication label;
generating an intermediate image by evaluating the captured image against the set of pre-processing rules; and
generating the medication information by evaluating the intermediate image against the set of word- or character-recognition rules.

5. The method of claim 1 wherein providing the medication information associated with the medication that the user intends to purchase comprises i) comparing the medication information to historical medication information associated with the user based on a set of interaction or reaction rules including rules that review historical adverse effects that the user experienced based on similar medication or rules that determine if the user is currently taking other medication that could adversely interact with the medication, ii) generating an alert based on detecting one or more medication conflicts associated with the comparison based on a set of severity rules, and iii) identifying one or more medication alternatives.

6. (canceled)

7. The method of claim 1 wherein the providing the medication information associated with the medication that the user intends to purchase further comprises:
providing the one or more recommended medication alternatives to a medical provider that prescribed the medication associated with the medication information based on a set of replacement rules;
providing alternative medication information; and
replacing the medication information with the alternative medication based on a set of dosage rules.

8. The method of claim 1, further comprising:
generating an image of a body;
obtaining a user selection of a portion of the image of the body;
obtaining symptom information associated with the selected portion of the image of the body;
generating one or more candidate illnesses associated with the symptom information and the selected portion of the image of the body based on a set of diagnosis rules;
selecting one or more medical providers based on a set of selection rules and the one or more candidate illnesses.

9. The method of claim 8 wherein generating one or more candidate illnesses further comprises:
obtaining location information associated with the user based on a set of locator rules; and
generating the one or more candidate illnesses based on an analysis of the obtained symptom information associated with the selected portion of the image of the body based on the obtained symptom or illness statistical information and a set of correlation rules .

10. (canceled)

11. The method of claim 1 wherein accessing the drug supply chain intermediary API to obtain medication price information further comprises:
obtaining location information associated with the user based on a set of locator rules; and
obtaining medication price information based on the provided medication information, a geographical region associated with the obtained location information, and a set of filter rules.

12. The method of claim 1 wherein identification information associated with the user is obfuscated or omitted from the notification to the PBM based on a set of anonymizing rules.

13. The method of claim 1 wherein displaying the medication purchase options comprises:
analyzing historical medication information associated with the user based on a set of filter rules;
analyzing insurance plan information associated with the insurance plan associated with the user and the PBM based on a set of threshold rules; and
recommending one or more medication purchase options in the displayed medication purchase options based on a set of modeling rules.

14. The method of claim 13 wherein the set of filter rules are used to drive an artificial intelligence computer algorithm that determines typical annual medication or healthcare consumption of the user.

15. The method of claim 13 wherein the set of modeling rules are used to drive an artificial intelligence computer algorithm that recommends medication purchase options.

16. (canceled)

17. (canceled)

18. The method of claim 1, further comprising:
accessing an approval API to obtain new treatment information associated with one or more symptoms or illnesses that are associated with the medication that the user intends to purchase based on a set of filter rules; and
notifying the user of the new treatment information.

19. The method of claim 1, further comprising:
drug supply chain intermediary API to obtain updated price information associated with the provided medication information based on a set of chronic-use rules; and
notifying the user of a price change based on the updated price information and a set of trend rules.

20. (canceled)

21. (canceled)

22. The method of claim 1, further comprising:
obtaining an emergency request for medical information associated with the user;
evaluating the request based on a set of authentication rules; and
providing one or more portions of the requested medical information based on the evaluated request.

23. The method of claim 1, further comprising:
evaluating the user selection of the medication purchase option based on a set of adherence rules.

24. The method of claim 23 wherein providing the historical evaluation information to the DDP comprises obfuscating or omitting identification information associated with the user based on a set of anonymizing rules.

25. A computing system for improving electronic communication in a healthcare environment comprising a plurality of heterogeneous computing devices, comprising:
a processor;
a memory;
a healthcare advisor module that is stored in the memory and that is configured, when executed by the processor, to:
receive from a computing device associated with a user an indication of a medication that the user intends to purchase;
determine and provide medication information associated with the indicated medication, including determining a target entity for purchase of the medication;
access a discount drug provider application programming interface (“API”) by electronically communicating with a drug discount program operated computing system to obtain medication price information based on the provided medication information, the obtained medication price information including a discount price associated with purchase of the medication from a discount drug provider and a discount drug provider (“DDP”) medication price associated with an insurance plan that is associated with the user and a DDP system;
generate a medication price information data object based on the provided medication information and the obtained medication price information, the medication price information data object having a plurality of fields that each have one or more values;
electronically send a notification to the system by electronically communicating with a computing device associated with the DDP system that the user is considering purchasing the medication for the discount price from an entity that is outside of the insurance plan associated with the user and the DDP system;
obtain a revised DDP medication price in near real-time from the DDP system based on the sent notification;
automatically generate and provide in near real-time revised medication price information by revising one or more values of one or more fields in the medication price information data object based on the revised DDP medication price;
provide medication purchase options to the user based on the generated revised medication price information and the determined target entity;
receive from the user an indication of a selected medication purchase option from the presented medication purchase options; and
access a pharmacy API by electronically communicating with a pharmacy operated computing system to provide the medication information to a pharmaceutical entity based on the indicated medication purchase option.

26. (canceled)

27. A computer-readable storage medium containing instructions for controlling a computer processor to perform a method comprising:
under control of a first computing device,
receiving an indication of medication that a user intends to purchase;
determining and providing medication information associated with the indicated medication;
accessing a drug supply chain intermediary application programming interface (“API”) of computing instructions executing on a second computing device distinct from the first computing device and operated by a first external third party to obtain medication price information based on the provided medication information, the obtained medication price information including a discount price associated with purchase of the medication from one or more drug supply chain intermediaries and a discount drug provider (“DDP”) medication price associated with a plan that is associated with the user and a DDP system;
generating a medication price information data object based on the provided medication information and the obtained medication price information, the medication price information data object having a plurality of fields that each have one or more values;
in near real-time,
notifying the DDP system through a third computing device distinct from and external to the first computing device that the user is considering purchasing the medication for the discount price from an entity that is outside of the plan associated with the user and the DDP system;
obtaining a revised DDP medication price from the DDP system based on the notification to the DDP system, the revised DDP medication price indicating a more competitive price than the discount price;
automatically generating revised medication price information, by revising one or more values of one or more fields in the medication price information data object, based on the revised DDP medication price; and
presenting medication purchase options to the user based on the generated revised medication price information and the determined target entity;
receiving from the user an indication of a selected medication purchase option from the presented medication purchase options;
accessing a pharmacy API of computing instructions executing on a fourth computing device distinct from the first computing device and operated by a second external third party to provide the medication information to a pharmaceutical entity based on the indicated medication purchase option.
